(19) United States
(12) Patent Application Publication
(10) Pub. No.: US 2026/0220869 A1
(43) Pub. Date: Jul. 30, 2026

(54) COMPUTER-IMPLEMENTED METHOD, DEVICE, SYSTEM AND COMPUTER PROGRAM PRODUCT FOR PROCESSING ANATOMIC IMAGING DATA

(71) Applicant: UNIVERSITÄT ZÜRICH, CH-8006 ZÜRICH (CH)

(72) Inventors: Hooman ESFANDIARI, Vancouver (CA); Philipp FÜRNSTAHL, Eglisau (CH); Mazda FARSHAD, Zumikon (CH); Sascha JECKLIN, Igis (CH)

(73) Assignee: UNIVERSITÄT ZÜRICH, CH-8006 ZÜRICH (CH)

(21) Appl. No.: 19/149,220

(22) PCT Filed: Feb. 5, 2024
(86) PCT No.: PCT/EP2024/052800

§ 371 Date: Jul. 18, 2025

(30) Foreign Application Priority Data
Feb. 6, 2023 (CH) 000102/2023

(51) Int. Cl. G06T 15/00; A61B 34/20; G06V 10/54; G16H 30/20; G16H 30/40

(52) U.S. Cl. CPC G06T 15/00; A61B 34/20; G06V 10/54; G16H 30/20; G16H 30/40

(57) ABSTRACT

A computer-implemented method for processing anatomic imaging data, the method comprising, for a plurality of anatomic specimens: acquiring 3D imaging data of a specific body part of an anatomic specimen of the plurality of anatomic specimens; acquiring 2D images of the specific body part of the anatomic specimen from a plurality of different perspectives with respect to the specific body part; determining a plurality of camera matrices P corresponding to the acquired 2D images; generating synthetic 2D images by projecting the acquired 3D imaging data in accordance with the plurality of camera matrices P; and associating the acquired 2D images with one of the synthetic 2D images as training data pairs in accordance with the plurality of camera matrices P and plurality of perspectives. The method further comprising generating a training dataset for use in training an artificial intelligence algorithm using the training data pairs.

### BACKGROUND OF THE INVENTION

### Field of the Invention

The present disclosure relates to a computer-implemented method, device, system and computer program product for processing anatomic imaging data. In particular, the present disclosure relates to a computer-implemented method, device, system and computer program product for processing anatomic imaging data, comprising generating a training dataset for use in training an artificial intelligence algorithm. Furthermore, the present disclosure relates to a computer-implemented method, device, system and computer program product for processing anatomic imaging data, comprising training of an artificial intelligence algorithm using the training dataset. Furthermore, the present disclosure relates to a computer-implemented method, device, system and computer program product for processing anatomic imaging data comprising processing acquired 2D images of a specific body part of an anatomic subject using the artificial intelligence algorithm.

The present disclosure further relates to a computer-implemented method of assisting positioning of a tool, such as a surgical tool, with respect to a specific body part of a patient. The present disclosure further relates to a computing device configured to assisting positioning of a tool, such as a surgical tool, with respect to a specific body part of a patient. The present disclosure even further relates to a system for assisting positioning of a tool with respect to a specific body part of a patient. The present disclosure even further relates a computer program product, comprising instructions, which, when carried out by a processing unit of a computing device, cause the computing device to assisting positioning of a tool, such as a surgical tool, with respect to a specific body part of a patient.

### Discussion of Related Art

Processing of anatomic imaging data, in particular processing of anatomic imaging data for intraoperative navigation and guidance of complex orthopedic interventions, remains a challenging task to date despite the existence of modern Computer Assisted Surgery (CAS) solutions. This is reflected in recent reports that have estimated the clinical adoption rate of CAS solutions to be on the order of 11% and 5% of the surgeries performed. This becomes particularly sobering in light of the documented evidence on the benefit of CAS solutions in improving the surgical accuracy and outcome. The relatively low clinical adoption rate for CAS solutions has been historically attributed to many factors ranging from the added surgical time and operational costs to line-of-sight issues (specific to solutions that require optical tracking systems) and most importantly the requirement for patient registration.

Patient registration, which is the process of aligning a preoperatively generated surgical plan to the patient's anatomy, has been addressed in the prior-art using a variety of algorithms such as feature-based registration, intensity-based registration, Statistical Shape Modelling (SSM) and more recently, data driven Artificial Intelligence registration methods. Despite the availability of advanced algorithms for patient-registration, commercially available methods for processing anatomic imaging data, in particular CAS solutions, generally rely on rudimentary approaches such as landmark-based methods. This is due to the fact that in spite of their low level of autonomy and versatility, such basic methods can be rather easily integrated into a CAS pipeline, in contrast to advanced registration methods that suffer from limiting factors such as: small capture range, lack of large training datasets, lack of robust similarity metrics for multi-modality registration and long computation time.

This has been the underlying reason behind the advent of algorithms that move toward providing registration-free alternatives for processing anatomic imaging data. In the context of orthopedic surgery, such methods generally rely only on ubiquitously available 2D imaging data, e.g. in the form of C-arm fluoroscopy. As an essential component, these alternative solutions need to process anatomic imaging data, such as reconstruct an anatomic subject's anatomy directly solely based on 2D imaging data, e.g. C-arm imaging. Registration-free CAS can be achieved either through full intraoperative Cone-Beam Computed Tomography (CBCT) or by more advanced algorithms that can reconstruct the 3D shape of the patient's anatomy using sparse fluoroscopy data. Registration-free CAS solutions that are based on intraoperative CBCT imaging have not been commonly adopted due to reasons such as the added cost, time and ionizing radiation.

Artificial intelligence-based algorithms designed for processing anatomic imaging data, in particular anatomical 3D reconstructions based on X-ray input, do not suffer from the aforementioned limitations thanks to their capability in processing anatomic imaging data, in particular reconstructing the patient's anatomy based on a small number of ubiquitously available 2D images, in particular intraoperative X-rays. These artificial intelligence-based data-driven methods can be seen as a stepping stone toward the development of registration-free methods for processing anatomic imaging data, such as CAS systems of the future.

However, the major technical bottleneck in creating such methods is the limited availability of large-scale annotated anatomic imaging. Among different factors, ethical and radiation exposure concerns along with the manual effort needed for data annotation are the underlying hurdles in collecting the required in-vivo training datasets. To surmount these hurdles, majority of the existing work opt for creating synthetic 2D datasets (e.g. X-ray) that can be generated together with the corresponding annotations in an automatic fashion. These synthetic datasets are commonly created using Digitally Reconstructed Radiograph (DRR) techniques that can simulate the projection of a planar X-ray based on an input CT volume with varying degrees of realism. Despite the flexibility and scalability of such datasets, they may fail in capturing the realistic anatomic imaging conditions such as the actual radiological properties of the anatomy, the noise characteristics of the imaging device and the back-scattering behaviour of the real 2D imaging environment. If used to train downstream artificial intelligence models such as 3D reconstruction networks, these synthetic datasets will inevitably condition the models to perform well only on the synthetic training data, while failing to achieve similar levels of performance when applied on real (acquired) 2D images (e.g. X-ray images). This is commonly referred to as the domain gap phenomenon.

### SUMMARY OF THE INVENTION

It is an object of the present disclosure to provide a computer-implemented method, a computing device respectively a computer program product for processing anatomic imaging data, comprising generating a training dataset for training an artificial intelligence algorithm, which do not have at least some of the disadvantages of the prior art.

In particular, it is an object of embodiments disclosed herein to provide a computer-implemented method, a computing device respectively a computer program product for processing anatomic imaging data, using a training dataset comprising paired real-synthetic X-ray images that can be acquired from anatomic specimens.

According to the present disclosure, these objects are addressed by the features of the independent claims. In addition, further advantageous embodiments follow from the dependent claims and the description.

This object is in particular addressed by a computer-implemented method for processing anatomic imaging data, the method comprising performing the following steps for a plurality of anatomic specimens: acquiring 3D imaging data of a specific body part of an anatomic specimen of the plurality of anatomic specimens (referred to as acquired 3D imaging data); acquiring 2D images of the specific body part of the anatomic specimen from a plurality of different perspectives with respect to the specific body part (referred to as acquired 2D images); determining a plurality of camera matrices P corresponding to the acquired 2D images, whereby the camera matrices P are indicative at least of the perspective of the 2D image with respect to the specific body part; generating synthetic 2D images by projecting the acquired 3D imaging data in accordance with the plurality of camera matrices P; and associating the acquired 2D images with one of the synthetic 2D images as training data pairs in accordance with the plurality of camera matrices P. The method further comprises generating a training dataset for use in training an artificial intelligence algorithm, for processing anatomic imaging data, using the training data pairs. In particular, generating a training dataset using the training data pairs comprises storage of the data pairs with the corresponding metadata and/or data indicative of the association of the acquired 2D images with the synthetic 2D image.

According to embodiments, one or more of the anatomic specimens are cadaveric, in particular human cadaveric specimens. Alternatively, or additionally, one or more of the anatomic specimens are living, in particular human living specimens (persons).

According to embodiments, the step of acquiring 3D imaging data of a specific body part of an anatomic specimen of the plurality of anatomic specimens comprises capturing, by a 3D imaging device, such as a computed tomography CT imaging device, a specific body part of an anatomic specimen. Alternatively, or additionally, the step of acquiring 3D imaging data of a specific body part of an anatomic specimen of the plurality of anatomic specimens comprises receiving, by a computing device, 3D imaging data (capturing a specific body part of an anatomic specimen) from a communicatively connected 3D imaging device. Alternatively, or additionally, the step of acquiring 3D imaging data of a specific body part of an anatomic specimen of the plurality of anatomic specimens comprises retrieving, by a computing device, 3D imaging data from a communicatively connected database, storing 3D imaging data of a plurality of anatomic specimens.

According to embodiments, the step of acquiring 2D images of the specific body part of the anatomic specimen from a plurality of different perspectives with respect to the specific body part comprises capturing, by a 2D imaging device, 2D images of the specific body part of the anatomic specimen from a plurality of different perspectives with respect to the specific body part. Alternatively, or additionally, the step of acquiring 2D images of the specific body part of the anatomic specimen from a plurality of different perspectives with respect to the specific body part comprises receiving, by a computing device, the 2D images from a communicatively connected 2D imaging device. Alternatively, or additionally, the step of acquiring 2D images of the specific body part of the anatomic specimen from a plurality of different perspectives with respect to the specific body part comprises retrieving, by a computing device, the 2D images from a communicatively connected database, storing 2D images of the specific body part of the anatomic specimens from a plurality of different perspectives with respect to the specific body part.

Determining a plurality of camera matrices P corresponding to the acquired 2D images comprises determining intrinsic and extrinsic camera properties of the acquired 2D images. Intrinsic camera properties relate to properties characterizing the 2D imaging device that captured the 2D images, such as focal length and, principal point location on the image plane. Extrinsic camera properties relate to the location and orientation of the camera with respect to the specific body part, in other words its perspective.

Having determined the camera matrices P corresponding to the acquired 2D images, indicative at least of the perspective of the 2D image with respect to the specific body part, synthetic 2D images are generated, by the computing device, by projecting the acquired 3D imaging data in accordance with the plurality of camera matrices P. In other words, the synthetic 2D images are generated as if there were captured by the same 2D imaging device and from the same perspective (same camera matrix) as the acquired 2D images. The technical considerations behind this approach is that it is close to impossible to precisely match camera matrices P with a prescribed camera matrix (of a synthetic 2D image) when acquiring 2D images of anatomic specimens due to unavoidable inaccuracy of the intrinsic and extrinsic camera properties. Instead of attempting to correct the inaccuracy of setting the intrinsic and extrinsic camera properties when capturing 2D images of anatomic specimens, according to the present disclosure, the generation of the synthetic 2D images, which does not suffer from such inaccuracies, is performed according to the camera matrices of the acquired 2D images.

In other words, instead of attempting to position the 2D images' device to match the camera matrix, in particular the perspectives of synthetic 2D images, the 2D images are synthetized in accordance with the camera matrices P of acquired 2D images when generating training data pairs.

The training dataset generated using the training data pairs is specifically adapted for use in training an artificial intelligence algorithm for processing anatomic imaging data. The training dataset is generated considering specific technical considerations to provide training data pairs whereby the acquired 2D images have been associated with synthetic 2D images in accordance with the corresponding camera matrices P. In particular, one or more of the acquired 2D images is associated with a synthetic 2D image generated with the corresponding camera matrix P, in particular with a corresponding perspective. In this way, the training data pairs (comprising associated acquired and synthetic 2D images) are not only suitable but specially adapted for use in training an artificial intelligence algorithm in that each data pair comprises an acquired 2D image to be provided as input and a synthetic 2D image as reference for expected/desired output for the artificial intelligence algorithm, or the other way around, i.e. a synthetic 2D image as input and an acquired 2D image as reference for expected/desired output. By providing corresponding data pairs, the artificial intelligence algorithm can be trained by applying a suitable objective function, cost function or lost function comparing a generated output with the desired output.

In order to allow a precise determination of the plurality of camera matrices P corresponding to the acquired 2D images and to allow projection of the acquired 3D imaging data in accordance with the plurality of camera matrices P, according to embodiments, the anatomic specimen is provided (fitted) with calibration device(s) before acquiring the 3D imaging data and the 2D images of the specific body part of the anatomic specimen. In case of living anatomic specimens, a registration device is used as calibration device which is non-invasively or minimally invasively attached to the anatomic specimens. In case of ex-vivo, cadaveric anatomic specimens, fiducial devices are attached as calibration device(s) to the anatomic specimens.

Having provided the anatomic specimen with calibration device(s), the 2D coordinates of the calibration device(s) are determined (referred to as determined 2D coordinates) in each of the 2D images.

Furthermore, having provided the anatomic specimen with calibration device(s), the 3D coordinates of the calibration device(s) are determined based on the 3D imaging data. Thereafter, a 2D-3D coordinate correspondence between the 2D coordinates of the calibration device(s) in each of the 2D images and the 3D coordinates of the calibration device(s) is determined. The plurality of camera matrices P corresponding to the acquired 2D images are determined further using the 2D-3D coordinate correspondence. In other words, by determining the coordinates of the calibration device(s) both in the acquired 2D and 3D images, the camera matrices P can be precisely determined and the 3D image data can be projected to produce synthetic 2D images precisely matching the camera matrices P of the acquired 2D images.

According to embodiments, in order to avoid the calibration device(s) being captured in the 3D imaging data and hence projected onto the synthetic 2D images, 3D imaging data is further acquired before providing the anatomic specimen with the calibration device(s). In order to compensate eventual alignment errors, a 3D-3D registration of the 3D imaging data acquired before providing the anatomic specimen with the calibration device(s) and the 3D imaging data acquired after providing the anatomic specimen with the calibration device(s) is performed by the computing device. After the 3D-3D registration of the 3D imaging data acquired before providing the anatomic specimen with the calibration device(s) and the 3D imaging data acquired after providing the anatomic specimen with the calibration device(s), the synthetic 2D images are generated by projecting the 3D imaging data acquired before providing the anatomic specimen with the calibration device(s) in accordance with the plurality of camera matrices P. In this way, the calibration device(s) are not present in the synthetic 2D images (but only the anatomic specimen).

While the calibration device(s) are beneficial for determining the camera matrices P of the acquired 2D images, the presence of the calibration device(s) in the acquired 2D images is undesired. Hence, in order to remove the calibration device(s) being captured in the 2D imaging data, according to embodiments, projections of the calibration device(s) in the 2D images are inpainted from the acquired 2D images before their use in generating the training dataset.

According to embodiments, determining the plurality of camera matrices P, corresponding to the acquired 2D images, comprises detecting 2D coordinates of the calibration device(s) on the acquired 2D images; detecting 3D coordinates of the calibration device(s) on the acquired 3D imaging data; determining a 2D-3D coordinate correspondence between the 2D coordinates and 3D coordinates of the calibration device(s); and determining the camera matrices P using the 2D coordinates and 3D coordinates of the calibration device(s) and the 2D-3D coordinate correspondence. In particular, determining the 2D-3D coordinate correspondence between the 2D coordinates and 3D coordinates of the calibration devices is performed in a repetitive process, whereby for each possible correspondence between the 2D coordinates and 3D coordinates of the calibration devices: the camera matrices P are estimated; the 3D imaging data is projected according to the estimated camera matrices P; and a re-projection error is calculated by comparing the projected 3D imaging data with the corresponding acquired 2D images. After having calculated the re-projection error for all possible correspondences between the 2D and the 3D coordinates of the calibration devices, the 2D-3D coordinate correspondence with the lowest re-projection error is used to determine the camera matrices P of the acquired 2D images.

According to embodiments, the computer-implemented method of the present disclosure further comprises acquiring a pre-training dataset for processing anatomic imaging data comprising a plurality of synthetic 2D images capturing the specific body part of anatomic specimens and pre-training an artificial intelligence algorithm (referred to as pre-trained artificial intelligence algorithm) using the pre-training dataset for processing anatomic imaging data. In view of the limited availability of annotated anatomic imaging, according to a particular embodiment, the pre-training dataset comprises synthetic 2D images, such as fluoroscopy shots (i.e., DRRs), generated with different camera matrices, in particular from different perspectives given input 3D image data, such as a CT scan. For example, using this method, up to several hundred annotated “synthetic” 2D images (capturing the specific body part) can be generated from a single annotated CT scan, which are annotated “synthetic” 2D images that can be used to train the artificial intelligence algorithm for processing anatomical images, such as reconstructing accurate anatomical 3D shapes from as few 2D intraoperative images as possible.

In light of the limitations and disadvantages of the domain gap as described in the background section of this application, it is a further object of embodiments disclosed herein to provide a domain adaptation framework method using which one can employ artificial intelligence algorithms that have been pre-trained solely on synthetic images for processing acquired (real) 2D images (e.g. X-rays). For example, such a method can be used for reconstructing an anatomical 3D shape (AS) of a specific body part of an anatomic subject based on 2D images of the specific body part and data indicative of camera matrix, in particular of the perspectives corresponding to the 2D images; and/or for anatomical segmentation of 2D images of the specific body part of an anatomic subject.

This object is achieved by applying a transfer learning process, namely training the pre-trained artificial intelligence algorithm (also referred to as re-training) using the training dataset comprising data pairs of synthetic and acquired (real) 2D images of corresponding camera matrices P.

According to embodiments, training the pre-trained artificial intelligence algorithm (also referred to as re-training) comprises affixing a first subset of network parameters, such as weights and biases of an artificial neuronal network, for network components of the pre-trained artificial intelligence algorithm and adjusting a second subset of network parameters using the training data pairs of the training dataset as input. For a particular embodiment of reconstructing an anatomical 3D shape a specific body part of an anatomic subject, re-training the pre-trained artificial intelligence algorithm comprises affixing network parameters, such as weights and biases of an artificial neuronal network, for network components of the pre-trained artificial intelligence algorithm except a 2D feature extractor module, and adjusting network parameters of the 2D feature extractor module using the training data pairs of the training dataset as input.

As a practical application, according to the present disclosure, the computer implemented method further comprises acquiring 2D images of the specific body part of an anatomic subject and processing the acquired 2D images using the artificial intelligence algorithm trained using the training dataset.

In this context, the term “anatomic subject” refers to a particular subject of interest, such as a patient during surgery or medical examination, as opposed to the plurality of “anatomic specimens” analysed to obtaining the training dataset.

Alternatively, the objective of providing a domain adaptation framework is addressed by a style transfer process, using which, one can employ artificial intelligence algorithms that have been pre-trained on synthetic images for processing acquired (real) 2D images (e.g. X-rays). In particular, the objective of providing a domain adaptation framework is addressed, according to embodiments, in that the computer-implemented method further comprises training a style transfer algorithm, using the training dataset; and applying a style transfer process onto acquired 2D images using the style transfer algorithm. The style transfer process is applied to the acquired 2D images (referred to as pre-conditioned acquired 2D images) before the processing of the acquired 2D images of an anatomic subject using the pre-trained artificial intelligence algorithm.

In other words, the domain gap (pre-training performed using vastly available but synthetic 2D images) is filled in that the input images (acquired 2D images of an anatomic subject) are pre-conditioned by a style transfer algorithm before being fed as input into the pre-trained artificial intelligence algorithm.

Specifically, the style transfer process comprises extracting texture information from synthetic 2D images of the training dataset; and transferring the texture information onto the acquired 2D image of the anatomic subject, while conserving underlying semantic content of the acquired 2D images. In other words, the acquired 2D images (of an anatomic subject) are made to look like synthetic 2D images, which the pre-trained artificial intelligence algorithm has been trained to process.

Unique to the style transfer process of the present disclosure is the development of models that translate acquired 2D images of an anatomic subject (e.g. real X-ray data) into synthetic 2D images (e.g. synthetic DRR images). In comparison to known approaches of transferring synthetic 2D images (e.g. synthetic DRR images) into acquired-like (e.g. realistic X-ray-like) images where the target domain is highly heterogeneous, the reverse transfer direction of the present disclosure is implemented to ensure a homogeneous target domain (e.g. by controlling the synthetic DRR space). Additionally, for a variety of applications where an artificial intelligence algorithm pre-trained on synthetic data is already available (e.g., segmentation, reconstruction, landmark detection and etc.), one can use the style transfer process of the present disclosure in a sequential fashion where the acquired 2D image (e.g. real X-ray) input is first transferred into the synthetic domain, which is in turn fed into the pre-trained artificial intelligence algorithm.

In particular embodiments, the style transfer process implements one or more of the following methods:
an Encoder-Decoder Deep Convolutional Network EDDC;
a Neural Style Transfer NST algorithm;
a Deep Convolutional Generative Adversarial Network DCGAN;
Cycle-Consistent Generative Adversarial Network CycleGAN.

As a practical application, according to the present disclosure, the computer implemented method further comprises acquiring 2D images of the specific body part of an anatomic subject, applying a style transfer process onto acquired 2D images using the style transfer algorithm, and processing the pre-conditioned acquired 2D images using a pre-trained artificial intelligence algorithm.

As a side note, in the context of the present disclosure, the terms “pre-training”, “pre-trained” refer to a training of an artificial intelligence algorithms using training data other than the training dataset generated as part of the present disclosure.

As a first use case, according to embodiments of the present disclosure, processing the acquired 2D images of the specific body part of the anatomic subject using the artificial intelligence algorithm comprises reconstructing an anatomical 3D shape of the specific body part of the anatomic subject, based on the acquired 2D images of the specific body part and data indicative of perspectives corresponding to the acquired 2D images.

As a further use case, according to embodiments of the present disclosure, processing the acquired 2D images of the specific body part of the anatomic subject using the artificial intelligence algorithm comprises anatomically segmenting the acquired 2D images to identify one or more anatomic characteristics of the specific body part.

The present application further relates to a computing device comprising a processing unit configured to carry out the method according to one of the embodiments disclosed herein. According to embodiments, the computing device comprises any one or a combination of: a central processing unit CPU, a physical or virtual computer, a local and/or remote portable, desktop and/or server computer, a cloud-based server or software as a service, and/or a dedicated hardware circuitry, such as an ASIC or FPGA.

The present application further relates to a system comprising a computing device according to one of the embodiments disclosed herein and an imaging device, such as a pre-, post- and/or intra-operative imaging device communicatively connected with the computing device and configured to capture 2D images of the specific body part of an anatomic subject.

The present application further relates to a computer program product, comprising instructions, which, when carried out by a processing unit of a computing device, cause the computing device to carry out the method according to any one of the embodiments disclosed herein.

According to embodiments the present disclosure, the training dataset, the pre-trained artificial intelligence algorithm, the trained artificial intelligence algorithm, and/or the style transfer algorithm are generated respectively trained for a specific body part. Correspondingly, their use for training respectively processing acquired 2D images is intended for the same specific body part.

It is a further object of the present disclosure to provide a method, computing device, system, and computer program product for assisting positioning of a tool with respect to a specific body part of a patient which overcome one or more of the disadvantages of the prior art.

In particular, it is an object of the present disclosure to provide a registration-free method for assisting positioning of a tool with respect to a specific body part of a patient that can reconstruct an anatomical 3D shape and generate a visual representation of a position of the tool with respect to the specific body part(s) of a patient using only intraoperative 2D imaging data, i.e. without the need for navigation hardware to be installed in the operating room.

According to the present disclosure, this object is addressed by the features of the independent claims. In addition, further advantageous embodiments follow from the dependent claims and the description.

In particular, this object is achieved by a computer-implemented method of assisting positioning of a tool, such as a surgical tool (e.g. a surgical drill, knife or surgical laser equipment) or a medical diagnosis tool with respect to a specific body part of a patient, the method comprising:
receiving intraoperative 2D imaging data;
reconstructing an anatomical 3D shape using the intraoperative 2D imaging data and using an artificial intelligence algorithm corresponding to the specific body part;
estimating a current position of the tool based on the intraoperative 2D imaging data; and
generating positioning guidance data comprising a visual representation of the estimated current position of the tool with respect to the anatomical 3D shape of the specific body part(s).

In particular embodiments, the steps of receiving intraoperative 2D imaging data; estimating a current position of the tool and generating guidance data are carried out repeatedly or continuously for a period of time in preparation of/preceding a surgical treatment of the patient.

### Receiving Intraoperative 2D Imaging Data

Intraoperative 2D imaging data is received by a computing device from an intraoperative imaging device arranged in the proximity of the patient. The intraoperative imaging device being arranged in the proximity of the patient hereby refers to a positioning allowing the intraoperative imaging device to capture the intraoperative 2D imaging data of the patient. The imaging data comprises a plurality of intraoperative 2D images. Two or more of the intraoperative 2D images capture the specific body part of the patient from two or more different perspectives with respect to the specific body part of the patient. One or more of the same plurality of intraoperative 2D images capturing the specific body part also capture at least a part of the tool from at least one perspective. In the context of the present disclosure, the term perspective, with respect to a perspective of the imaging data, refers to a location (e.g. in an x, y, and z Cartesian coordinate system) and/or orientation (e.g. roll, pitch, yaw) of the intraoperative imaging device relative to the specific body part, respectively relative to the tool.

According to embodiments of the present disclosure, the intraoperative 2D imaging data comprises one or more of: a) radiation-based images, in particular X-ray image(s); b) ultrasound image(s); c) arthroscopic image(s); d) optical imagery; and/or e) any other cross-sectional imagery.

Images of the specific body part of the patient respectively a part of the tool are captured using an intraoperative imaging device communicatively connected to the computing device. In case of radiation-based images, the intraoperative imaging device may comprise a C-arm intraoperative imaging device based on X-ray technology. The C-arm intraoperative imaging device comprises a generator (X-ray source) and an image intensifier or flat-panel detector. A C-shaped connecting element allows movement horizontally, vertically and/or around a swivel axes, so that 2D X-ray images of the patient can be produced from various perspectives around the patient. The generator emits X-rays that penetrate the patient's body. The image intensifier or detector converts the X-rays into a visible image that is transmitted to the computing device.

The intraoperative 2D imaging data comprises data indicative of perspectives corresponding to the plurality of the intraoperative 2D images, identifying the location and/or orientation of the intraoperative imaging device that captures plurality of the intraoperative 2D images, such as a location in an x, y, and z Cartesian coordinate system and/or orientation as roll, pitch, yaw of the intraoperative imaging device relative to the specific body part. According to embodiments disclosed herein, the data indicative of the perspectives corresponding to the plurality of the intraoperative 2D images is stored in a datastore comprised by or communicatively connected to the computing device. Alternatively, or additionally, the perspectives corresponding to the intraoperative 2D imaging data are estimated by the computing device based on the intraoperative 2D imaging data.

In a particular embodiment, estimating perspectives corresponding to the intraoperative 2D imaging data is performed using a tool geometrical model indicative of a geometry of the tool. First, a plurality of projections of the tool geometrical model are computed from a plurality of candidate perspectives. Candidate perspectives are selected as discrete perspectives within a predefined space of possible perspectives of the intraoperative imaging device. In other words, non-realistic locations and orientations of the intraoperative imaging device relative to the patient are not considered to conserve computing power. Thereafter, the perspectives corresponding to the intraoperative 2D images of the intraoperative 2D imaging data are identified by comparing the at least part of the tool as captured by the respective 2D image of the intraoperative 2D imaging data with the plurality of projections computed from the plurality of candidate perspectives. In particular, the comparison comprises applying a matching function to identify a best match between one of the computed projections of the “virtual” tool (based on the tool geometrical model) from the plurality of candidate perspectives and the part of the “physical” tool as captured by the intraoperative imaging device. The candidate perspective producing the best match is selected as the estimated perspective.

According to embodiments disclosed herein, estimating perspectives corresponding to the intraoperative 2D imaging data is performed using an artificial intelligence algorithm trained using a multitude of imaging data sets with known perspectives. In order to overcome the limitations of the availability and/or accuracy of imaging data sets with known perspectives, a multitude of imaging data sets comprising intraoperative 2D images from known perspectives are generated from 3D imaging data, in particular computed tomography CT scans. Using this artificial intelligence algorithm, trained prior to the surgery, the intraoperative position of the intraoperative imaging device can be estimated only based on the intraoperative images without requiring neither an external tracking device nor a calibration phantom.

### Generating an Anatomical 3D Shape

An anatomical 3D shape of the specific body part is reconstructed by the computing device using an artificial intelligence algorithm corresponding to the specific body part based on the intraoperative 2D imaging data and data indicative of the perspectives corresponding to the plurality of the intraoperative 2D images.

According to embodiments disclosed herein, the anatomical 3D shape is reconstructed as a voxelized volume and/or mesh. It is important to emphasize that the artificial intelligence algorithm must be a model corresponding to the specific body part, enabling a reconstruction of a 3D anatomical shape from a plurality of intraoperative 2D images of the specific body part.

According to particular embodiments disclosed herein, the artificial intelligence algorithm is trained using a multitude of annotated imaging data sets capturing body parts (of persons potentially other than the patient) corresponding to the specific body part of the patient. The annotations of the imaging data sets comprise data identifying and/or describing properties of the body part, such as identifying pixels, vectors, contours, surfaces within intraoperative 2D images and/or voxels capturing the specific body part within 3D images capturing the specific body part.

In order to overcome the limitations of the availability and/or accuracy of annotated imaging data sets capturing body parts corresponding to the specific body part of the patient, according to embodiments disclosed herein, a multitude of annotated imaging data sets are generated from annotated 3D imaging data, in particular computed tomography CT scans, capturing body parts corresponding to the specific body part of the patient. In particular, synthetic intraoperative 2D images, such as fluoroscopy shots (i.e., DRRs), are generated from different perspectives around the patient given an input preoperative CT scan. For example, using this method, up to several hundred annotated “synthetic” intraoperative 2D images (capturing the specific body part) can be generated from a single annotated CT scan, which are annotated “synthetic” intraoperative 2D images that can be used by the artificial intelligence algorithm to improve its capabilities of reconstructing accurate anatomical 3D shapes from as few 2D intraoperative images as possible.

### Estimating a Current Position of the Tool

Having reconstructed the anatomical 3D shape, a current position of the tool with respect to the anatomical 3D shape of the specific body part is estimated based on the intraoperative 2D imaging data, in particular intraoperative 2D images of the imaging data capturing of the tool. According to embodiments disclosed herein, estimating the current position of the tool is performed using a tool geometrical model indicative of a geometry of the tool. First, a projection of the tool geometrical model is compared with the at least part of the tool as captured by the respective 2D image of the intraoperative 2D imaging data. The tool geometrical model being projected onto the plane(s) of one or more of the intraoperative 2D images of the intraoperative 2D imaging data capturing the at least part of the tool. The plane of the intraoperative 2D images of the intraoperative 2D imaging data is determined based on the perspective of each 2D image. Thereafter, a position of the tool geometrical model that produces a projection onto the planes of the intraoperative 2D images of the intraoperative 2D imaging data is determined that (best) matches the at least part of the tool as captured by the respective 2D image of the intraoperative 2D imaging data. In other words, the reverse process is applied as compared to the (initial) determination of the perspectives of the intraoperative 2D imaging data. However, this reverse process is applied not necessarily on the same intraoperative 2D images (of the intraoperative 2D imaging data) as the intraoperative 2D images used for determining the perspectives of the images used for reconstruction of the anatomical 3D shape.

According to embodiments disclosed herein, while the anatomical 3D shape is reconstructed once, in an initial stage of the method of assisting positioning of the tool, the estimation of the current position of the tool is carried out repeatedly at set intervals and/or triggered by certain events and/or manually triggered.

In order to improve the accuracy of estimating the position of the tool and/or to improve the accuracy of estimating perspectives corresponding to the intraoperative 2D imaging data, according to further embodiments, the method of the present disclosure further comprises providing a tool in accordance with a tool geometrical model. Wherein the tool geometrical model is specifically designed to optimize the estimation of its position based on as few intraoperative images as possible. In particular, the tool is designed such that at least a part thereof is not completely rotationally symmetric around any of the axis of the Cartesian coordinate system in order to allow estimation of the tool's orientation based on intraoperative 2D images. Alternatively, or additionally, the tool is designed to comprise special markers to facilitate its identification based on 2D intraoperative images.

### Generating Positioning Guidance Data

Having reconstructed the anatomical 3D shape of the specific body part and having estimated the current position of the tool, positioning guidance data is generated by the computing device, comprising a visual representation of the estimated current position of the tool with respect to the anatomical 3D shape of the specific body part(s). According to embodiments disclosed herein, the guidance data is generated as a 2D image to be displayed on a computer display. Alternatively, or additionally, the guidance data is generated as an augmented reality overlay, comprising overlay metadata allowing an augmented reality device, such as a headset, to project the overlay onto the field of view of a user such that the overlay is aligned with the user's view of the specific body part of the patient and/or aligned with the user's view of the tool. According to embodiments disclosed herein, the visual representation of the estimated current position of the tool is overlaid onto a visual representation of the reconstructed anatomical 3D shape.

According to embodiments disclosed herein, the computing device controls a display device to display at least part of the guidance data, the display device is a computer screen, an augmented reality headset, or any device configured to display guidance data.

In order to guide surgeons to correctly position the tool, according to further embodiments disclosed herein, a prescribed position of the tool with respect to the anatomical 3D shape of the specific body part is identified by the computing device and a visual representation of the prescribed position of the tool is overlaid onto a visual representation of the estimated current position of the tool. The prescribed position of the tool is retrieved or received by the computing device from a datastore comprised by or communicatively connected to the computing device. Alternatively, or additionally, the prescribed position of the tool is computed by the computing device, the prescribed position of the tool being determined by an optimization function based on the anatomical 3D shape of the body part(s) as well as data indicative of a surgical procedure.

Embodiments disclosed herein are advantageous, as they enable automatically performing a pre-surgical planning based on reconstructed anatomical 3D shapes and guiding surgeons in placement of surgical tool(s). Neither a preoperative planning stage is required to define the safe implant trajectories nor is a registration of preoperative data to the intraoperative patient's position is needed, given that the intraoperative 2D imaging data is used to reconstruct the anatomical 3D shape of the body part (e.g. spine), based on which prescribed positions/trajectories of the tool(s) can be identified.

It is a further object of the present disclosure to provide a computing device for positioning of a tool with respect to a specific body part of a patient that can reconstruct an anatomical 3D shape and generate a visual representation of a position of the tool with respect to the specific body part(s) of a patient using only intraoperative 2D imaging data, i.e. without the need for navigation hardware to be installed in the operating room and without the need for a registration process of a preoperative plan.

The above-identified object is further achieved by a computing device comprising: a data input interface; a data output interface; a processing unit; and a storage unit. The data input interface, such as a wired (e.g. Ethernet, DVI, HDMI, VGA) and/or wireless data communication interface (e.g. 4G, 5G, Wifi, Bluetooth, UWB) is communicatively connectable with an intraoperative imaging device and configured to receive intraoperative 2D imaging data therefrom. The data output interface such as a wired (e.g. Ethernet, DVI, HDMI, VGA) and/or wireless data communication interface (e.g. 4G, 5G, Wifi, Bluetooth, UWB, infrared) is configured to transmit at least part of guidance data to a display device communicatively connectable to the data output interface. The storage unit comprising instructions, which, when carried out by the processing unit, causes the computing device to carry out the method of assisting positioning of a tool according to any one of the embodiments disclosed herein.

According to embodiments, the computing device is a stand-alone computer communicatively connected to the intraoperative imaging device. Alternatively, or additionally, the computing device is a remote computer (e.g. a cloud-based computer) communicatively connected to the intraoperative imaging device using a communication network, in particular at least partially using a mobile communication network. Alternatively, or additionally, the computing device is integrated into the intraoperative imaging device or the display device.

It is a further object of the present disclosure to provide a system for positioning of a tool with respect to a specific body part of a patient that can reconstruct an anatomical 3D shape and generate a visual representation of a position of the tool with respect to the specific body part(s) of a patient using only intraoperative 2D imaging data, i.e. without the need for navigation hardware to be installed in the operating room and without the need for a registration process of a preoperative plan.

The above-identified object is further achieved by a system comprising: a computing device according to any one of the embodiments disclosed herein; an intraoperative imaging device; and a display device, the system being configured to carry out the method according to any one of the embodiments disclosed herein. The intraoperative imaging device is communicatively connected to the computing device and arranged in the proximity of the patient allowing the intraoperative imaging device to capture the intraoperative 2D imaging data of the patient such that two or more of the intraoperative 2D images capture the specific body part of the patient from two or more different perspectives with respect to the specific body part of the patient. One or more of the same plurality of intraoperative 2D images capturing the specific body part also capture at least a part of the tool from at least one perspective. In case of radiation-based images as intraoperative images, the intraoperative imaging device comprises a C-arm intraoperative imaging device based on X-ray technology. The C-arm intraoperative imaging device comprises a generator (X-ray source) and an image intensifier or flat-panel detector. A C-shaped connecting element allows movement horizontally, vertically and/or around a swivel axes, so that 2D X-ray images of the patient can be produced from various perspectives around the patient. The generator emits X-rays that penetrate the patient's body. The image intensifier or detector converts the X-rays into a visible image that is transmitted to the computing device. The display device is a computer screen, an augmented reality headset, or any device configured to display a guidance data.

It is a further object of the present disclosure to provide a computer program product for positioning of a tool with respect to a specific body part of a patient that can reconstruct an anatomical 3D shape and generate a visual representation of a position of the tool with respect to the specific body part(s) of a patient using only intraoperative 2D imaging data, i.e. without the need for navigation hardware to be installed in the operating room and without the need for a registration process of a preoperative plan.

The above-identified object is addressed by a computer program product, comprising instructions, which, when carried out by a processing unit of a computing device, cause the computing device to carry out the method according to any one of the embodiments disclosed herein.

According to embodiments, the instructions (comprised by the computer program product) comprise an artificial intelligence algorithm corresponding to a specific body part of a patient, the artificial intelligence algorithm having been trained using a multitude of annotated imaging data sets capturing body parts corresponding to the specific body part of the patient, wherein the annotations comprise data identifying and/or describing properties of the body part.

According to embodiments, the instructions (comprised by the computer program product) comprise instructions to control an intraoperative imaging device to capture intraoperative 2D imaging data comprising intraoperative 2D images, a plurality of the intraoperative 2D images capturing the specific body part of the patient from a plurality of different perspectives with respect to the specific body part of the patient and one or more of the plurality of the intraoperative 2D images capturing at least a part of the tool from at least one perspective.

According to embodiments, the instructions (comprised by the computer program product) comprise instructions to control a display device such as to display at least part of the guidance data comprising a visual representation of the estimated current position of the tool, a visual representation of the reconstructed anatomical 3D shape and/or a visual representation of the prescribed position of the tool.

It is to be understood that both the foregoing general description and the following detailed description present embodiments and are intended to provide an overview or framework for understanding the nature and character of the disclosure. The accompanying drawings are included to provide a further understanding and are incorporated into and constitute a part of this specification. The drawings illustrate various embodiments, and together with the description serve to explain the principles and operation of the concepts disclosed.

The term “particular” as used in the present specification refers to embodiments of the disclosure, without any indication of preference or indication that features introduced as particular would be essential to all embodiments of the disclosure.

### BRIEF DESCRIPTION OF SEVERAL VIEWS OF THE DRAWINGS

The present disclosure will be explained in more detail, by way of example, with reference to the drawings.

FIG. 1 shows a flowchart illustrating steps of a method for processing anatomic imaging data according to the present disclosure, comprising generating a training dataset for use in training an artificial intelligence algorithm;

FIG. 2 shows a flowchart illustrating steps of a process of determining camera matrices;

FIG. 3 shows a data collection and processing pipeline of a specific application of a method for processing anatomic imaging data according to the present disclosure on fresh-frozen ex-vivo full torso specimens;

FIG. 4 shows illustrative 2D images during a process of inpainting calibration devices from 2D images (of the specific application of FIG. 3);

FIG. 5 shows a flowchart illustrating steps of a method for processing anatomic imaging data by applying a transfer learning process, comprising generating a training dataset, pre-training of an artificial intelligence algorithm using a pre-training dataset, training the pre-trained artificial intelligence algorithm with the training dataset and processing anatomic imaging data using the trained artificial intelligence algorithm;

FIG. 6 shows an illustration of a specific embodiment of re-training of a pre-trained artificial intelligence algorithm by applying a transfer learning process;

FIG. 7 shows a flowchart illustrating steps of a method for processing anatomic imaging data by applying a style transfer process, comprising generating a training dataset, pre-training of an artificial intelligence algorithm using a pre-training dataset, training a style transfer algorithm using the training dataset, pre-conditioning anatomic imaging data using the style transfer algorithm and processing the pre-conditioned anatomic imaging data using the pre-trained artificial intelligence algorithm;

FIG. 8 shows a flowchart illustrating steps of applying a style transfer algorithm onto anatomic imaging data;

FIG. 9 shows illustration of a first embodiment of a style transfer algorithm based on a Deep Convolutional Generative Adversarial Network (DCGAN);

FIG. 10 shows illustration of a second embodiment of a style transfer algorithm based on a Cycle-Consistent Generative Adversarial Network (CycleGAN);

FIG. 11 shows a highly schematic perspective view of a system for assisting positioning of a tool as installed in an operating room, according to an embodiment of the present disclosure;

FIG. 12 shows a flowchart illustrating steps of a method of assisting the positioning of a tool, according to an embodiment of the present disclosure;

FIG. 13 shows a flowchart illustrating steps of reconstructing an anatomical 3D shape based on the intraoperative 2D imaging data and data indicative of the camera matrices corresponding to the plurality of the intraoperative 2D images, according to an embodiment of the present disclosure;

FIG. 14 shows a schematic illustration of segmenting intraoperative 2D imaging data in order to identify the specific body part of the patient;

FIG. 15 shows a schematic illustration of a further embodiment of segmenting intraoperative 2D imaging data, comprising identification of region(s) of interest followed by semantically segmenting the region(s) of interest in order to identify the specific body part of the patient within the region(s) of interest;

FIG. 16 shows a schematic illustration of the reconstruction of an anatomical 3D shape based on segmented intraoperative 2D imaging data using an artificial intelligence algorithm corresponding to the specific body part;

FIG. 17 shows a flowchart illustrating steps of a further embodiment of reconstructing an anatomical 3D shape in multiple stages;

FIG. 18 shows a schematic illustration of determining a prescribed position of the tool;

FIG. 19A shows an illustrative example of a visual representation of the estimated current position of the tool and a visual representation of the prescribed position of the tool overlaid onto a visual representation of the reconstructed anatomical 3D shape;

FIG. 19B shows an illustrative example of a visual representation of the estimated current position of the tool overlaid onto a 2D image of the intraoperative 2D imaging data ID; and

FIG. 19C shows an illustrative example of a visual representation of the estimated current position of the tool, a visual representation of the prescribed position of the tool and a visual representation of an ideal screw trajectory of a surgical implant onto a visual representation of the reconstructed anatomical 3D shape.

### DETAILED DESCRIPTION OF THE INVENTION

Reference will now be made in detail to certain embodiments, examples of which are illustrated in the accompanying drawings, in which some, but not all features are shown. Indeed, embodiments disclosed herein may be embodied in many different forms and should not be construed as limited to the embodiments set forth herein; rather, these embodiments are provided so that this disclosure will satisfy applicable legal requirements. Whenever possible, like reference numbers will be used to refer to like components or parts.

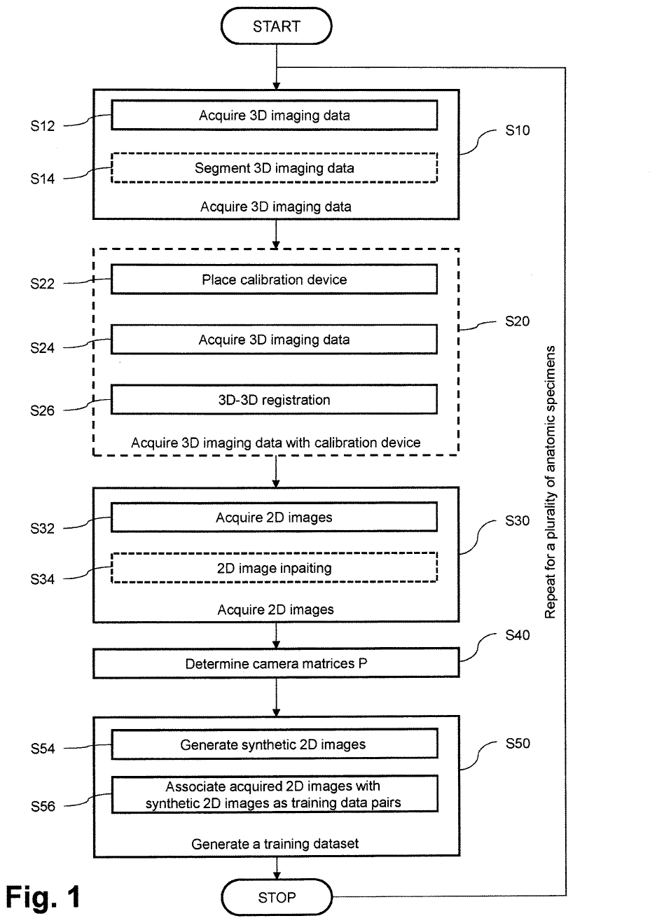

Steps of a method for processing anatomic imaging data according to the present disclosure, comprising generating a training dataset for use in training an artificial intelligence algorithm, are illustrated in FIG. 1.

In a first step S10, 3D imaging data of a specific body part of an anatomic specimen of the plurality of anatomic specimens (referred to as acquired 3D imaging data) is acquired. In a first substep S12 of step S10, 3D imaging data, in particular computed tomography CT scans, of a plurality of anatomic specimens are acquired using a 3D imaging device. In an optional substep S14 of step S10, individual body parts, such as vertebrae, are segmented in a 3D volume.

In a step S30, subsequent or simultaneous to step S10, 2D images are acquired. In a substep S32, 2D images of the specific body part of the anatomic specimen are acquired from a plurality of different perspectives with respect to the specific body part (referred to as acquired 2D images).

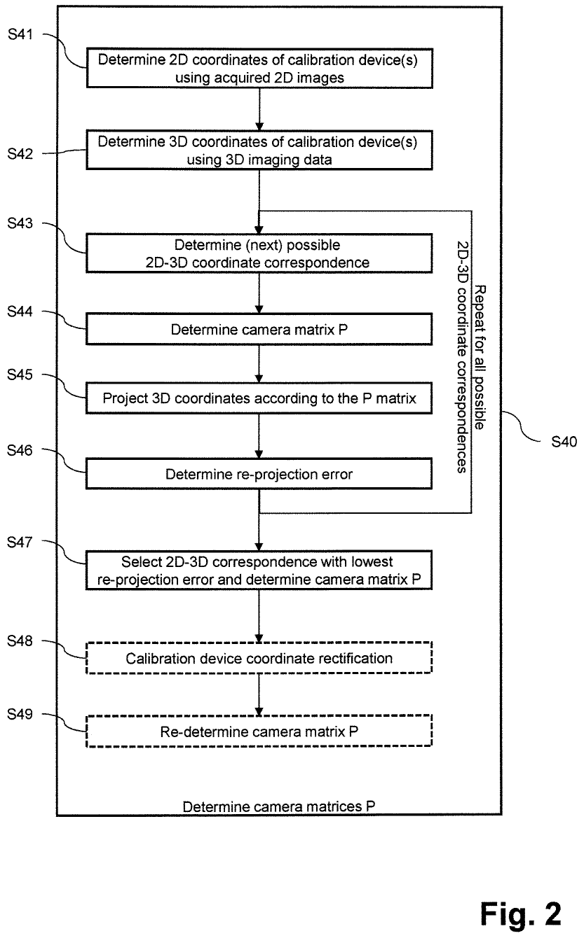

In step S40, a plurality of camera matrices P corresponding to the acquired 2D images are determined, whereby the camera matrices P are indicative at least of the perspective of the 2D images with respect to the specific body part. Details of determining the plurality of camera matrices P are described below with reference to FIG. 2. Finally, in a step S50, a training dataset is generated. In a first substep S52 of step S50, a plurality of camera matrices P corresponding to the acquired 2D images are determined by the computing device 10. As a further substep S54 of step S50 (generation of a training dataset), synthetic 2D images are generated, by the computing device, by projecting the acquired 3D imaging data in accordance with the plurality of camera matrices P. Hence, one synthetic 2D image is generated for each camera matrix P, i.e. for each acquired 2D image perspective. As a further substep S56 of step S50, the acquired 2D images are associated with one of the synthetic 2D images as training data pairs in accordance with the plurality of camera matrices P, in particular the plurality of perspectives. In other words, each of the 2D images is associated with a synthetic 2D image which has been generated according to a camera matrix P corresponding to the perspective of the acquired 2D image.

In an optional step S20, in order to allow a precise determination of the plurality of camera matrices P corresponding to the acquired 2D images (see step S30) and to allow projection of the acquired 3D imaging data in accordance with the plurality of camera matrices P, 3D imaging data is acquired with calibration devices attached to the anatomic specimen. In a substep S22 of step S20, the anatomic specimen is provided with a number n of calibration devices before acquiring the 3D imaging data and the 2D images of the specific body part of the anatomic specimen. In a substep S24, 3D imaging data of a specific body part of an anatomic specimen of the plurality of anatomic specimens (referred to as acquired 3D imaging data) is acquired with the calibration devices attached. In a substep S26, in order to compensate eventual alignment errors, a 3D-3D registration of the 3D imaging data acquired before providing the anatomic specimen with calibration device and the 3D imaging data acquired after providing the anatomic specimen with calibration device is performed by the computing device 10. After the 3D-3D registration of the 3D imaging data acquired before providing the anatomic specimen with calibration device and the 3D imaging data acquired after providing the anatomic specimen with calibration device, the synthetic 2D images are generated (in steps S54) by projecting the 3D imaging data acquired before providing the anatomic specimen with calibration device in accordance with the plurality of camera matrices P. In this way, the calibration devices are not present in the synthetic 2D images (but only the anatomic specimen).

In an optional substep S34 of step S30, in case the 2D images have been acquired with calibration devices attached, in order to remove the calibration devices being captured in the 2D imaging data, according to embodiments, projections of the calibration devices in the 2D images are inpainted/removed from the 2D images.

FIG. 2 shows substeps of the camera matrix P determination S40. In a first substep S41, 2D coordinates of the calibration devices are detected on the acquired 2D images, for example using a circular Hough transformation algorithm applied on thresholded 2D images (referred to as detected 2D coordinates). In a substep S42, the 3D coordinates of the calibration devices are determined (referred to as detected 3D coordinates) based on the 3D imaging data, for example by initial thresholding. Thereafter, in a sequence of repetitive substeps S43 to S46, all possible correspondences between the 2D and the 3D coordinates (referred to as 2D-3D coordinate correspondence) and the respective camera matrices P are determined, the 3D coordinates being projected and a re-projection error being calculated for each possible correspondences between the 2D and the 3D coordinates. In the first repetitive substep S43, the (next) possible 2D-3D coordinate correspondence of the calibration devices is selected. Subsequently, in substep S44, the camera matrices P corresponding to the acquired 2D images are determined based on the currently selected 2D-3D coordinate correspondence, for example using a Direct Linear Transform algorithm. Following determination of the camera matrices P for the currently selected 2D-3D coordinate correspondence, in a substep S45, the 3D imaging data (captured after the provision of the calibration devices) is projected in accordance with the plurality of camera matrices P. The projected 3D imaging data is overlayed with the corresponding acquired 2D images thereby calculating a so-called re-projection error-substep S46. This sequence of substeps S43 to S46 is repeated for all possible correspondences between the 2D and the 3D coordinates. The number of possible correspondences between the 2D and the 3D coordinates is results from the number n of calibration devices captured by both the 2D and 3D images, according to the formula of combinations:

$${}_{n} {C}_{2} = \frac{n !}{2 ( n - 2 ) !}$$

After having calculated the re-projection error for all possible correspondences between the 2D and the 3D coordinates, the 2D-3D coordinate correspondence with the lowest re-projection error is selected as correct, substep S47. This 2D-3D coordinate correspondence is then used to determine the camera matrices P for each of the acquired 2D images.

According to embodiments, whereby a relatively high number of calibration devices are provided in order to enable a more precise determination of the camera matrices P, only a first subset n′ of the calibration devices (referred to as reference calibration devices) is considered in the sequence of substeps S43 to S47 to limit the number of all possible correspondences between the 2D and the 3D coordinates. In this case the number n′, which defines the number of all possible correspondences, is the number of reference calibration devices.

Thereafter, having determined the 2D-3D coordinate correspondence of the reference calibration devices, in optional substep S48, all calibration devices (not only the reference calibration devices) are projected in accordance with the selected plurality of camera matrices P and estimated 2D coordinates of all calibration devices are determined from these projections. Based on a comparison of these estimated 2D coordinates with the detected 2D coordinates of substep S41, the 2D-3D coordinate correspondence of all calibration devices is determined allowing the camera matrix P to be re-calculated (in substep S49) leveraging all calibration devices. Hence, an optimum balance can be achieved between fitting of a high number of calibration devices, allowing precise camera matrices P determination, and the computing power needed to determine the re-projection error of each camera matrix P with all possible 2D-3D correspondences.

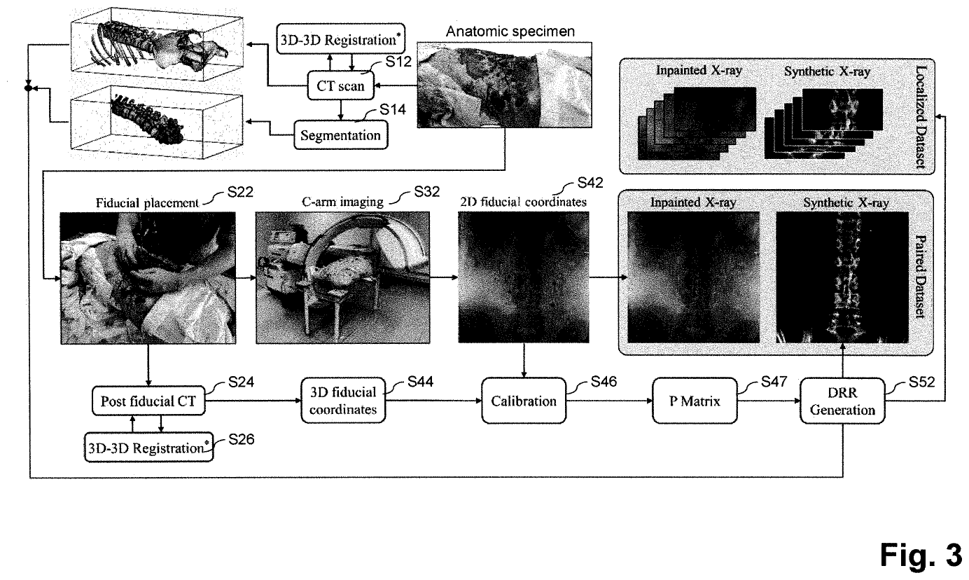

The data collection and processing pipeline of a specific application of a fresh-frozen ex-vivo full torso specimens as described in FIGS. 1, 2 and 4 before is illustrated in FIG. 3. The process starts: in a step S12, by acquiring a CT scan (e.g. with a slice thickness: 0.75 mm, in-plane resolution: 0.5 mm×0.5 mm) of a plurality of fresh-frozen ex-vivo full torso specimens, based on which segmentation of the individual body parts, such as vertebrae, is performed in the 3D volume, in a step S14.

Thereafter, in a step S22 stainless steel spherical fiducials (specific embodiments of a calibration device) are placed inside the frozen soft tissue, 7 fiducials with a 2.5 mm radius and 7 fiducials with a 1.5 mm radius, by first inserting K-wires (3 mm diameter) that indicate an intended drilling trajectory and using fluoroscopic guidance to visually verify the intended trajectories and if confirmed, making a subsequent drill to create a narrow tunnel inside the frozen tissue for placing the fiducials. After the fiducial insertion phase, the residual tunnel left in the frozen soft tissue may be sealed off using a super glue with a low viscosity. When placing the fiducials, it should be ensured that bony structures remain intact and the fiducials are inserted in an imaginary cylinder encapsulating the spinal column and covering an area from L1 to L5. This is to ensure that the fiducials have sufficient separation and are visible in clinically relevant imaging angles (AP, lateral and oblique).

Thereafter, in a step S24, a post-fiducial placement CT image is acquired from the anatomic specimen explicitly showing the relative placement of the fiducials with respect to the anatomy. A 3D-3D registration between the initial CT scan (fixed image) and the post-fiducial placement CT scan is then performed, in a step S26, followed by extracting, in a step S41, a list containing the 3D fiducial coordinates Xi∈R3, i∈[1, 2, . . . , 14] from this CT image through initial thresholding of the metallic objects and later performing a 3D connected component analysis.

In a further step S32, X-ray images of the anatomic specimen are collected, for example using a clinical-grade mobile C-arm device, starting from the Anterior-Posterior (AP) as the base viewing angle. Starting from the AP view, the specimen is imaged in 3° increments by rotating the C-arm gantry in the transverse plane until a ±102° orbit angle is achieved in both directions. Furthermore, starting from the AP view angle, the C-arm's tilt movement is used in 3° increments to image the specimen in the sagittal plane until the tilt angle of ±25° is reached in both directions resulting in oblique X-rays. Similarly, and starting from the lateral view angle, the tilt movement is used in 5° increments in the sagittal plane until a ±15° tilt is achieved. During the image acquisition phase, the C-arm gantry is positioned so that the X-rays are centered at L3 wile capturing the entire lumbar region as far as possible.

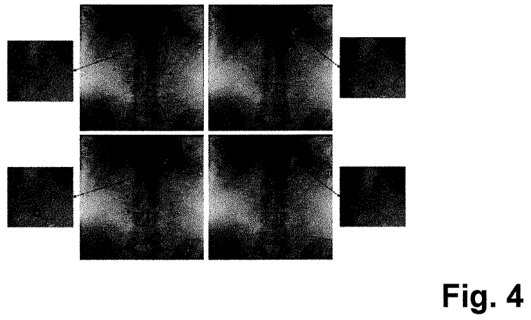

Once the X-rays have been acquired, a calibration process, step S43, is used to calibrate the X-rays using the coordinates of the projected fiducial markers in 2D and their corresponding 3D coordinates (details of the calibration process are described below with reference to FIG. 4).

The calculated camera matrix P is decomposed into extrinsic and intrinsic parameters by:

$$P = [ KR \vert - {KRX}_{o} ] \to M = KR ,$$

$$m = - {KRX}_{o}$$

$${X}_{o} = - {M}^{- 1} m$$

$$qr ( {M}^{- 1} ) = {R}^{T} {K}^{- 1} \to q = {R}^{T} ,$$

$$r = {K}^{- 1}$$

where K is the matrix containing the intrinsic camera parameters, R is the rotation matrix, Xo is the focal point coordinates, and qr is used to decompose a matrix into an orthogonal matrix q and an upper triangular matrix r.

Subsequently, in a substep S52 of step S50, using the recovered intrinsic and extrinsic camera parameters, a DRR image I is generated using the recovered intrinsic and extrinsic camera parameters using:

$$I ( p ) = \int A ( {T}^{- 1} L ( p , s ) ) ds$$

where I(p) is the intensity of the DRR at image point p, T=[R|X o]: R3→R3 is the transformation between the object and the image plane and L(p, s) is the ray initiating from the X-ray source and intersecting with the image plane at point p parameterized by s. Following this approach, the DRR image is projected from the same viewpoint as the acquired X-ray image to obtain paired synthetic-real X-ray data.

The entire aforementioned process (i.e., preoperative CT, fiducial placement, post fiducial CT, calibration and the downstream DRR generation) is performed for all the X-ray images collected from anatomic specimens. Using 3D segmentation masks, 2D masks on the DRR images are generated, using which synthetic-real X-ray image pairs are localized so that solely a single vertebral level is present on a given image.

As illustrated in FIG. 4, the process of determining the camera matrices P for the specific application of FIG. 3 is as follows:

### Fiducial Detection; Substep S41:

The 2D coordinates of the projected fiducials xj ∈R2, j∈[1, 2, . . . , 14] is first detected using a circular Hough transformation algorithm applied on thresholded images. This may be displayed as a suggestion to the user (FIG. 4a), who in turn could accept, reject or modify the detected points using keyboard and mouse inputs.

### Calculating 2D-3D Coordinate Correspondence; Substeps S43 to S47:

Up to this stage, the 2D and 3D fiducial coordinates were extracted, however, the correspondence between the two lists of coordinates x∈R2 and X∈R3 was not addressed. To this end, the beads with larger radii and their 3D coordinates as reference fiducials are considered, for which the corresponding 2D coordinates are desired in a first step. Assuming a given 2D-3D correspondence, a Direct Linear Transform (DLT) algorithm may be used to calculate the camera matrix P (containing the intrinsic and extrinsic imaging parameters) and the associated re-projection error. This re-projection error is then used as a cost function in a Random Sample Consensus (RANSAC) framework, whereby the calculation of the P matrix and the resultant re-projection error is repeated for all possible 2D-3D correspondences of the reference fiducials. The particular correspondence yielding the smallest re-projection error is considered as correct and is used to calculate camera matrix P, which is the first estimation of the camera matrix P (calculated only using the reference fiducials), substep S47. This camera matrix P is then used to project all the fiducial points from the 3D space X∈R3 to the 2D image space by: x=P X, where x∈R2 is the estimated 2D coordinates of the projected fiducials (FIG. 4b).

### Fiducial 2D-3D Coordinate Correspondence Rectification; Substep S48:

By comparing the estimated fiducial projections % and the detected fiducial projections x, the 2D-3D coordinate correspondence for all of the fiducials is recovered, allowing (re) calculating the camera matrix P, substep S49, leveraging all of the fiducial markers (FIG. 4c).

### Fiducial Inpainting; Step S34:

The projections of the fiducial markers are then inpainted on the acquired X-rays (FIG. 4d).

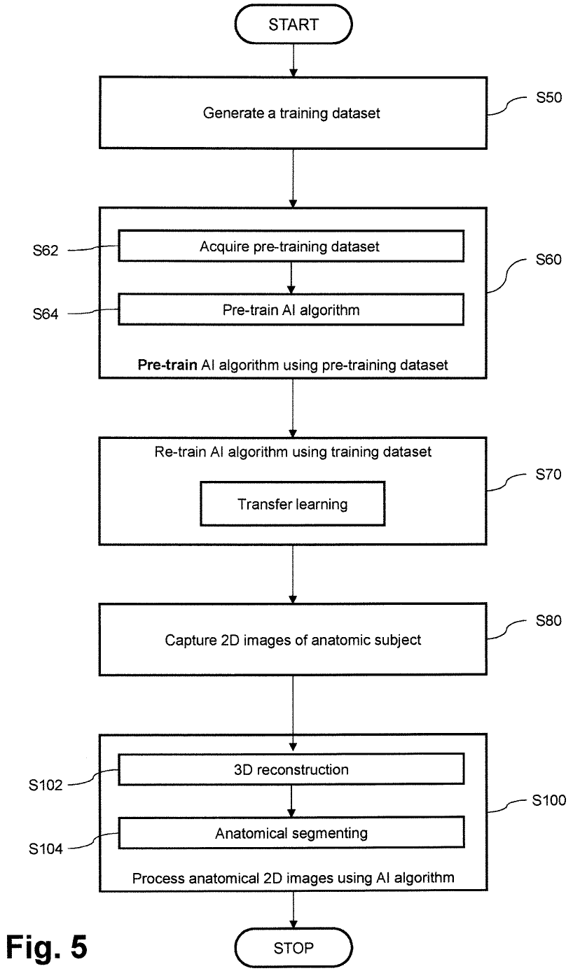

FIG. 5 shows a flowchart illustrating steps of a method for processing anatomic imaging data, applying a transfer learning process, comprising generating a training dataset, pre-training of an artificial intelligence algorithm using a pre-training dataset, training the pre-trained artificial intelligence algorithm with the training data pairs from the training dataset and processing anatomic imaging data using the trained artificial intelligence algorithm.

In a step S60, an artificial intelligence algorithm is pre-trained using a pre-training dataset. In a first substep S62, a pre-training dataset is acquired. In order to overcome the limitations of the availability and/or accuracy of annotated imaging datasets capturing body parts corresponding to the specific body part, a pre-training dataset comprising a multitude of imaging datasets generated from 3D imaging data, in particular computed tomography CT scans, capturing the specific body part(s). In particular, synthetic 2D images, such as fluoroscopy shots (i.e., DRRs), are generated with different camera matrices P, in particular from different perspectives given an input preoperative CT scan. For example, using this method, up to several hundred “synthetic” 2D images (capturing the specific body part) can be generated from a single CT scan. In a subsequent substep S64 of step S60, the artificial intelligence algorithm is pre-trained using the pre-training dataset.

Thereafter, in a step S70, the pre-trained artificial intelligence algorithm is (re-) trained using the training dataset generated according to one of the embodiments disclosed herein (described with reference to FIGS. 1 to 4). Training the pre-trained artificial intelligence algorithm (also referred to as re-training) comprises affixing a first subset of network parameters, such as weights and biases of an artificial neuronal network, for network components of the pre-trained artificial intelligence algorithm and adjusting a second subset of network parameters using the training data pairs of the training dataset as input. For a particular embodiment of reconstructing an anatomical 3D shape a specific body part of an anatomic subject, re-training the pre-trained artificial intelligence algorithm comprises affixing network parameters, such as weights and biases of an artificial neuronal network, for network components of the pre-trained artificial intelligence algorithm except a 2D feature extractor module, and adjusting network parameters of the 2D feature extractor module using the training data pairs of the training dataset as input.

As a next major step S80, 2D images of the specific body part of an anatomic subject, such as a patient during surgery or medical examination, are acquired.

Thereafter, in a step S100, the acquired 2D images are processed using the artificial intelligence algorithm first pre-trained using the pre-training dataset and then (re-) trained using the training dataset.

In a first (alternative or additional) substep S102 of step S100, processing the acquired 2D images of the specific body part of the anatomic subject using the artificial intelligence algorithm comprises reconstructing an anatomical 3D shape of the specific body part of the anatomic subject, based on the acquired 2D images of the specific body part and data indicative of camera matrices P, in particular of the perspectives corresponding to the acquired 2D images.

As a second (alternative or additional) substep S104, processing the acquired 2D images of the specific body part of the anatomic subject using the artificial intelligence algorithm comprises anatomically segmenting the acquired 2D images to identify one or more anatomic characteristics of the specific body part.

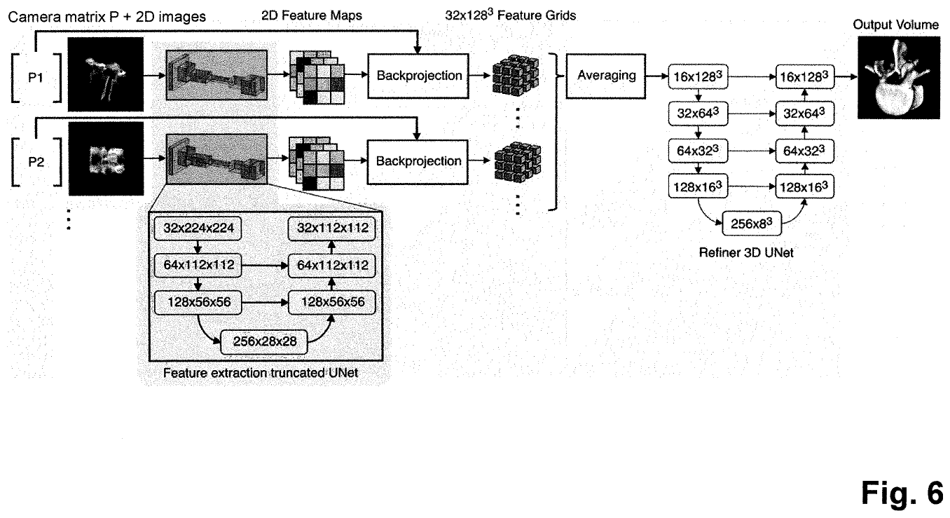

FIG. 6 shows a specific embodiment of re-training of a pre-trained artificial intelligence algorithm. Given that the artificial intelligence algorithm was pre-trained (and tested) using synthetic 2D images, the objective of re-training is to use the data pairs (of acquired 2D images and corresponding synthetic 2D images of the training dataset generated according to one of the embodiments disclosed herein) along with the recovered camera matrices P (i.e., extrinsic and intrinsic imaging parameters) to re-train the pre-trained artificial intelligence algorithm in a transfer learning fashion.

As shown on FIG. 6, the pre-trained artificial intelligence algorithm accepts localized 2D images as its input along with the corresponding imaging parameters. As a first step of the re-training process, 2D feature maps are extracted using a UNet model, to be later passed onto a differentiable backprojection module that uses the input imaging parameters (i.e., the camera matrix P) to create 3D feature grids. Later the 3D feature grids are averaged and fed into a refiner UNet model that in turn outputs the reconstructed 3D shape. During the transfer learning process, the pre-trained artificial intelligence algorithm is re-trained by affixing the network parameters (weights and biases) for all of the network components except the network parameters of the 2D feature extractor module. The trainable network parameters for the 2D feature extractor module are adjusted during the transfer learning process using the collected training data pairs as input.

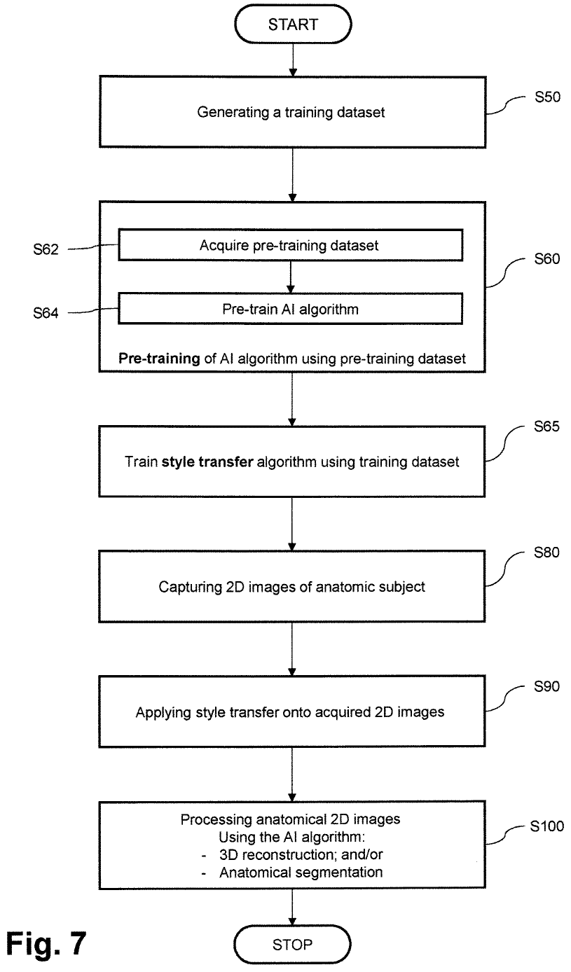

FIG. 7 shows a flowchart illustrating steps of a method for processing anatomic imaging data by applying a style transfer process, comprising generating a training dataset, pre-training of an artificial intelligence algorithm using a pre-training dataset, training a style transfer algorithm using the training dataset, pre-conditioning anatomic imaging data using the style transfer algorithm by applying a style transfer onto acquired 2D images and processing the pre-conditioned anatomic imaging data using the pre-trained artificial intelligence algorithm.

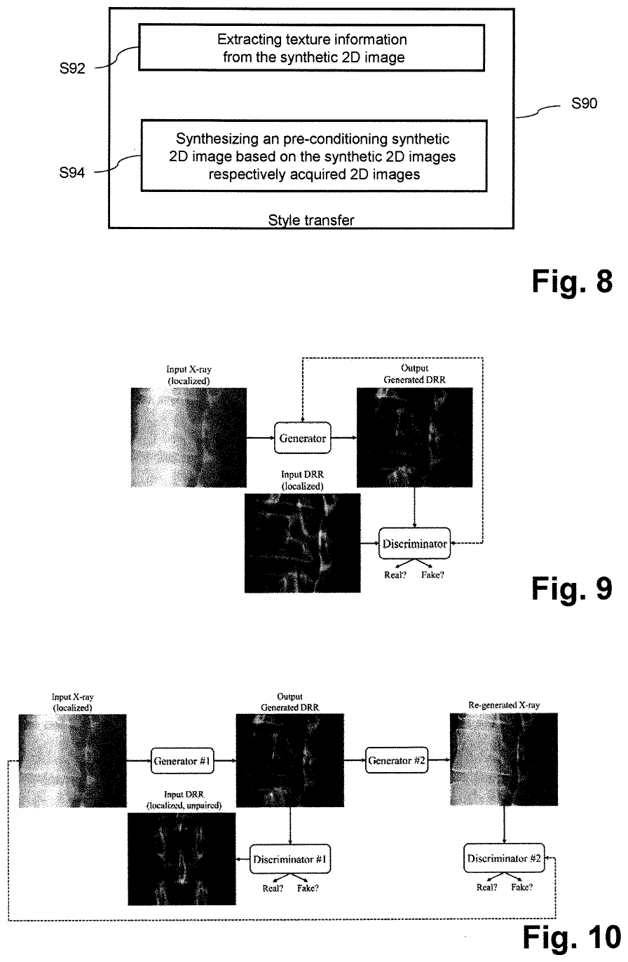

Alternatively, to the transfer learning approach, depicted in FIGS. 5 and 6, this approach, according to the embodiment shown on FIGS. 7 and 8, can be used. Therein, the provision of a domain adaptation framework is addressed by a style transfer process on acquired (real) 2D images, employing artificial intelligence algorithms that have been pre-trained on synthetic images. Similar to the description of transfer learning shown in FIG. 5, steps S50, generating a training dataset, and S60, pre-training an artificial intelligence algorithm using a pre-training dataset are performed. Then, instead of performing transfer learning as step S70 in FIG. 5, a style transfer learning algorithm is trained using the training dataset in step S65. Similar as in transfer learning, 2D images of an anatomic subject are captured in a step 70. Different to transfer learning shown in FIG. 5, the trained transfer learning algorithm is then applied to the acquired (or captured) 2D images in a step S90. Similar to the transfer learning, the anatomical 2d images are processed in a step S100, though in this case, compared to FIG. 5, with the captured 2d images that have been processed by the style transfer algorithm. In other words, instead of performing transfer learning (step S70), a style transfer algorithm is trained (S65) and the style transfer algorithm is applied onto acquired 2D images (S90). As a consequence, processing the anatomical 2D images (S100) is performed on pre-conditioned, enhanced 2D images.

In particular, in a step S65, a style transfer algorithm is trained using the training dataset. The style transfer algorithm is trained using the training dataset to be able to extract texture information from synthetic 2D images and transfer the texture information onto acquired 2D images, while conserving underlying semantic content of the acquired 2D images.

Having trained the style transfer algorithm, in step S90, the style transfer algorithm is applied onto the acquired 2D images (referred to as pre-conditioned acquired 2D images) before the processing of the acquired 2D images (using the artificial intelligence algorithm).

Step S90 of applying a style transfer algorithm is shown in more detail in FIG. 8. In a substep S92 of the style transfer process, texture information is extracted from synthetic 2D images of the training dataset. In a substep S94, a pre-conditioned acquired 2D image of the anatomic subject is synthetized by transferring the texture information onto the acquired 2D image while conserving underlying semantic content of the acquired 2D images.

Known methods of style transfer for intraoperative 2D imaging data generally tackle the transfer from synthetic 2D imaging (i.e., DRR) to acquired 2D imaging (i.e. X-ray domain), in hopes of achieving more realistic synthesized 2D images to be used for training an artificial intelligence algorithm. On the contrary, the key idea of the style transfer approach of the present disclosure is to apply the style transfer concept in the reverse direction to transfer the acquired (i.e. X-ray) 2D images into synthetic 2D imaging (i.e., DRR) domain for the following reasons;

Even under controlled imaging conditions, acquired (i.e. X-ray) 2D images have a high variation in image and radiological properties depending on factors such as: imaging technology, the detector type (intensifier such AS panel), device settings, patient's Body Mass Index (BMI), imaging view angle and back-scattering. This makes the task of synthetic to acquired 2D imaging (i.e. DRR to X-ray) transfer inherently challenging given that the target domain itself in this setup (i.e., the X-ray domain) is of high heterogeneity. In contrast, the synthetic (i.e. DRR) 2D images are created according to the present disclosure with a constant threshold applied on the underlying acquired 3D image (i.e. CT scan). The effect being that for example all the CT voxels with a Hounsfield value lower than the threshold were ignored during the DRR generation phase. This threshold was empirically selected to only project the bone structures on the synthetic (i.e. DRR) 2D images, which resulted in a homogeneous target domain.

Similar to other artificial intelligence-based methods that use synthetic (i.e. DRR) 2D images data for training purposes, the pre-trained artificial intelligence algorithm of the present disclosure was already pre-trained using a large number of annotated synthetic (i.e. DRR) 2D images. Therefore, by delivering an acquired (i.e. X-ray) to synthetic (i.e.DRR) style transfer algorithm, one can first convert the acquired (i.e. X-ray) 2D images into the synthetic (i.e. DRR) domain and then use the pre-trained artificial intelligence algorithm.

Using the training dataset (generated according to the method of the present disclosure), according to various embodiments, four different types of style transfer algorithms can be applied.

Encoder-Decoder Deep Convolutional Network (EDDC): This style transfer algorithm can be implemented using an hourglass encoder-decoder architecture. To train the EDDC network, the training dataset generated according to any of the embodiments of the present disclosure is used, comprising localized paired synthetic-real 2D images by considering the acquired (i.e. X-ray) 2D images as the network input and the corresponding localized synthetic (i.e. DRR) images as the network target.

Neural Style Transfer (NST): this algorithm operates by separating the style and the content from the input images using deep convolutional networks as a feature extractor. In order to generate the output image, the content information of the acquired (i.e. X-ray) image is extracted and combined with the style information abstracted from the synthetic (i.e. DRR) image. At the heart of this algorithm are two specialized loss functions, one tasked with estimating the content similarity between the generated image and the input acquired (i.e. X-ray) image (i.e., content loss) and the other responsible for estimating the style similarity between the generated image and the input synthetic (i.e. DRR) image. In order to train the NST, the localized paired synthetic-real training dataset generated according to any of the embodiments of the present disclosure is used.

Deep Convolutional Generative Adversarial Network (DCGAN): generative adversarial networks have widely been used for image synthesis tasks, when GAN models are trained to generate a new image based on random noise input. According to the present disclosure, as shown in FIG. 9, the GAN-based image synthesis task is converted into a style transfer task by swapping the random noise input with real/acquired (i.e. X-ray) 2D images. This can be accomplished thanks to the localized paired synthetic-real training dataset generated according to any of the embodiments of the present disclosure.

Cycle-Consistent Generative Adversarial Network (CycleGAN): in cases where only unpaired training data is available for style transfer purposes, CycleGAN networks are preferred that ensure reversible image-to-image transfer and include mechanisms for preventing mode collapse. In order to address this use case, the CycleGAN network architecture shown in FIG. 10 may be used, which includes two generator and two discriminator networks. Given that unpaired training data is sufficient for training such CycleGAN networks, the localized paired synthetic-real training dataset (generated according to any of the embodiments of the present disclosure) and additional larger synthetic (i.e. DRR) 2D imaging data dataset may be used. This may be performed to identify whether only unpaired synthetic-real data is sufficient for the desired style transfer task.

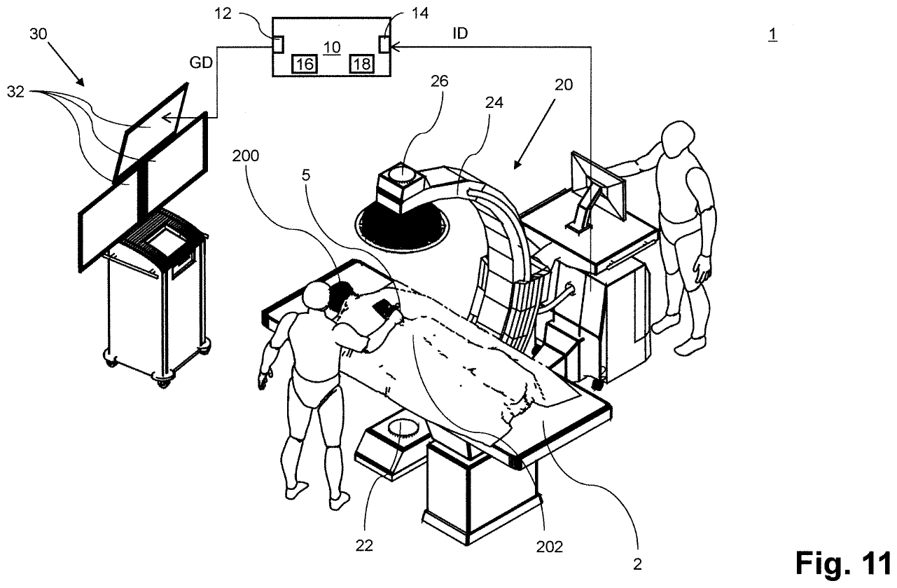

Turning now to FIGS. 11 to 19, a particular practical application of the method of generating training data respectively of training an artificial intelligence algorithm (reconstructing an anatomical 3D shape (AS) of a specific body part based on intraoperative 2D imaging data (ID) and data indicative of perspectives corresponding to the plurality of the intraoperative 2D images) of assisting positioning of a tool, such as a surgical tool, with respect to a specific body part of a patient shall be described.

FIG. 11 shows a highly schematic perspective view of a system 1 for assisting positioning of a tool 5 as installed in an operating room, the patient 200 laying on an operating table 2. As illustrated, the system 1 comprises a computing device 10; an intraoperative imaging device 20; and a display device 30. The system 1 is illustrated on the basis of an embodiment utilizing radiation-based images as intraoperative images. Accordingly, the intraoperative imaging device 20 comprises a C-arm intraoperative imaging device 20 capturing intraoperative images using X-ray technology. The C-arm intraoperative imaging device 20 comprises a generator (X-ray source) 22. A C-shaped connecting element (C-arm) 24 allows movement horizontally, vertically and/or around a swivel axes, so that 2D X-ray images of the patient 200 can be produced from various perspectives around the patient. The generator 22 emits X-rays that penetrate the patient's body 200. A detector 26 converts the X-rays into imaging data ID that is transmitted to the computing device 10.

The intraoperative imaging device 20 is communicatively connected to the computing device 10 and arranged in the proximity of the patient 200 allowing the intraoperative imaging device 20 to capture the intraoperative 2D imaging data ID of the patient 200 such that two or more of the intraoperative 2D images capture the specific body part 202 of the patient 200 from two or more different perspectives with respect to the specific body part 202 of the patient 200. One or more of the same plurality of intraoperative 2D images capturing the specific body part 202 also capture at least a part of the tool 5 from at least one perspective.

In the illustrated embodiment, the display device 30 comprises a series of computer screens 32 communicatively connected to the computing device 10 and configured to display guidance data GD.

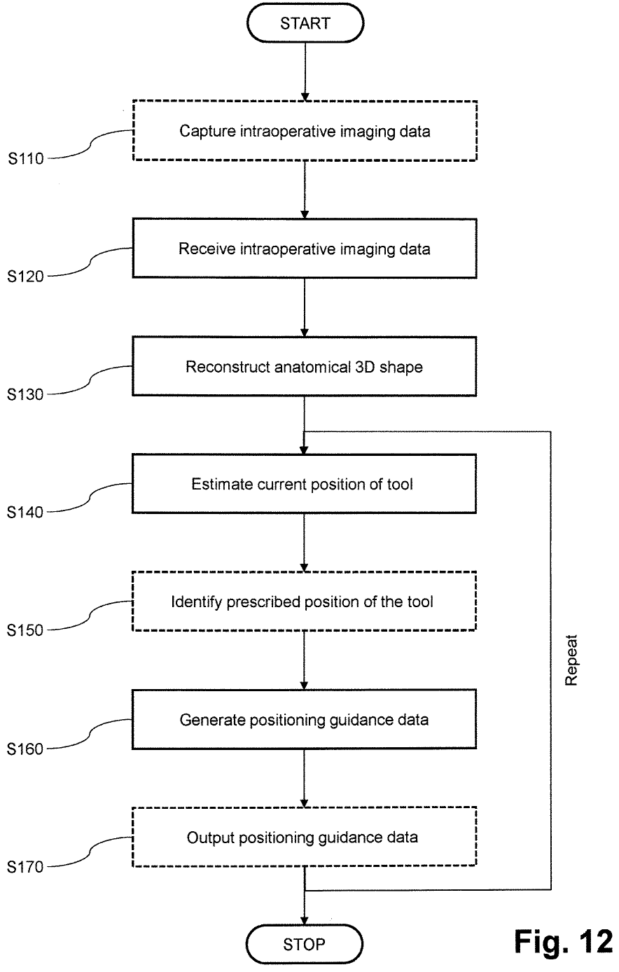

Turning now to the flowchart of FIG. 12, the steps of the computer-implemented method of assisting positioning of a tool 5 with respect to a specific body part 202 of a patient 200 shall be described.

As shown on FIG. 12, the method comprises the following major steps:
Step S110: capturing intraoperative 2D imaging data;
Step S120: receiving intraoperative 2D imaging data;
Step S130: reconstructing an anatomical 3D shape using the intraoperative 2D imaging data and using an artificial intelligence algorithm corresponding to the specific body part 202;
Step S140: estimating a current position 5c of the tool based on the intraoperative 2D imaging data ID;
Step S150: Identifying prescribed position 5p of the tool;
Step S160: reconstructing positioning guidance data GD comprising a visual representation of the estimated current position 5c of the tool 5 with respect to the anatomical 3D shape AS of the specific body part 202; and
Step S170: outputting the positioning guidance data GD using a display device 30.

Steps specific to particular embodiments are illustrated on the figures with dashed lines.

In a step S110, intraoperative 2D imaging data ID is captured by an intraoperative imaging device 20 arranged in the proximity of the patient 200. The intraoperative 2D imaging data ID comprises data indicative of perspectives corresponding to the plurality of the intraoperative 2D images, identifying the location and/or orientation of the intraoperative imaging device 20 that captures plurality of the intraoperative 2D images, such as a location in an x, y, and z Cartesian coordinate system and/or orientation as roll, pitch, yaw of the intraoperative imaging device 20 relative to the specific body part 202 of the patient 200.

According to a first embodiment, the data indicative of the perspectives corresponding to the plurality of the intraoperative 2D images is stored in a datastore comprised by or communicatively connected to the computing device 10. The perspectives stored in a datastore are determined by tracking the C-arm 24 to estimate the imaging parameters at the time of exposure, through which the intraoperatively acquired intraoperative 2D images can be assigned to their respective intrinsic and extrinsic imaging parameters that effectively define the perspectives from which the intraoperative 2D images have been generated from. Optionally, the tracking the C-arm 24 is preceded by a calibration process, a preoperative calibration process (i.e., pre-calibration), in which the C-arm 24 is maneuvered in a specific manner to cover the intented range-of-motion. During this pre-calibration phase, the mathematical relationship between the tracking observations and the imaging parameters is established at specific pose intervals, which will be later used to derive an interpolation function that can produce the intraoperative imaging parameters based on the tracking data.

Alternatively, or additionally, the perspectives corresponding to the intraoperative 2D imaging data ID are estimated by the computing device 10 based on the intraoperative 2D imaging data ID. In an embodiment, a calibration algorithm extracts the perspectives of the intraoperative 2D images by placing a precisely fabricated calibration object (i.e., phantom), which include distinct features (e.g. radiopaque features) with known geometry, in the imaging field and estimate the imaging parameters based on the projection of those features.

Alternatively, or additionally, estimating perspectives corresponding to the intraoperative 2D imaging data ID is performed using an artificial intelligence algorithm trained using a multitude of imaging data sets with known perspectives. In order to overcome the limitations of the availability and/or accuracy of imaging data sets with known perspectives, a multitude of imaging data sets comprising intraoperative 2D images from known perspectives are generated from 3D imaging data, in particular computed tomography CT scans. For example, simulated intraoperative fluoroscopy shots (i.e. Digitally Reconstructed Radiographs, DRRs) generated based on preoperative CT scans along with their corresponding pose parameters are used to train Convolutional Neural Networks (CNNs) for regression tasks. Using this artificial intelligence algorithm, trained prior to the surgery, the intraoperative position of the intraoperative imaging device 20 can be estimated only based on the intraoperative images without requiring neither an external tracking device nor a calibration phantom.

In a subsequent step S120, the intraoperative 2D imaging data ID is received by the computing device 10 from the intraoperative imaging device 20 via its data input interface 14.

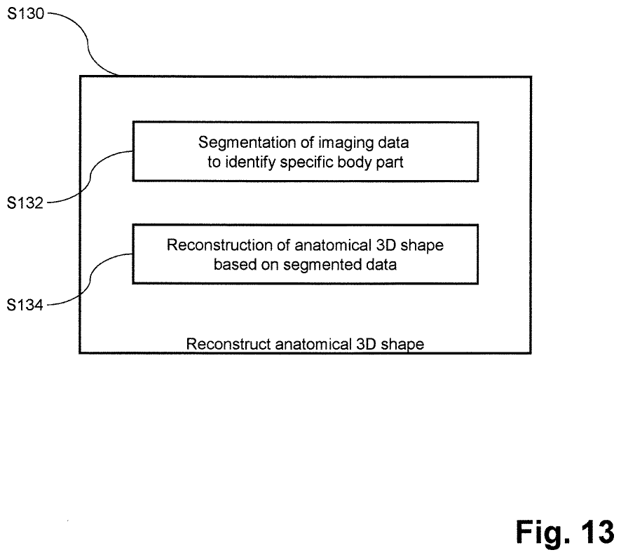

In subsequent step S130, an anatomical 3D shape AS of the specific body part 202 is reconstructed by the computing device 10 using an artificial intelligence algorithm corresponding to the specific body part 202 based on the intraoperative 2D imaging data ID and data indicative of the perspectives corresponding to the plurality of the intraoperative 2D images. A detailed description of step S130 of reconstructing an anatomical 3D shape AS is provided with reference to FIGS. 13, 14, 15, 16 and 17.

In step S140, the current position 5c of the tool 5 with respect to the anatomical 3D shape AS of the specific body part 202 is estimated based on intraoperative 2D images of the imaging data ID capturing of the tool 5. The current position 5c of the tool 5 is performed based on prior knowledge of the geometry of the tool 5 as described by a tool geometrical model. First, a projection of the tool geometrical model is compared with at least a part of the tool 5 as captured by the respective 2D image of the intraoperative 2D imaging data ID. The tool geometrical model being projected onto the plane(s) of one or more of the intraoperative 2D images of the intraoperative 2D imaging data ID capturing at least a part of the tool 5. The plane of the intraoperative 2D images of the intraoperative 2D imaging data ID is determined based on the perspective of each 2D image. Thereafter, a position of the tool geometrical model that produces a projection onto the planes of the intraoperative 2D images of the intraoperative 2D imaging data ID is determined that (best) matches the at least part of the tool 5 as captured by the respective 2D image of the intraoperative 2D imaging data ID.

According to embodiments disclosed herein, while the anatomical 3D shape is reconstructed once, in an initial stage of the method of assisting positioning of the tool, the estimation of the current position 5c of the tool 5 is carried out repeatedly at set intervals and/or triggered by certain events and/or manually triggered.

In order to improve the accuracy of estimating the position of the tool 5, a tool 5 is proved in accordance with a tool geometrical model. Wherein the tool geometrical model is specifically designed to optimize the estimation of its position based on as few intraoperative 2D images as possible. In particular, the tool is designed such that at least a part thereof is not completely rotationally symmetric around any of the axis of the Cartesian coordinate system in order to allow estimation of the tool's orientation based on intraoperative 2D images. Alternatively, or additionally, the tool is designed to comprise special markers to facilitate its identification based on 2D intraoperative images.

In step S150, a prescribed position 5p of the tool 5 with respect to the anatomical 3D shape AS of the specific body part 202 is identified by the computing device 10 and a visual representation of the prescribed position 5p of the tool 5 is overlaid onto a visual representation of the estimated current position 5c of the tool 5 in order to assist surgeons to correctly position the tool 5.

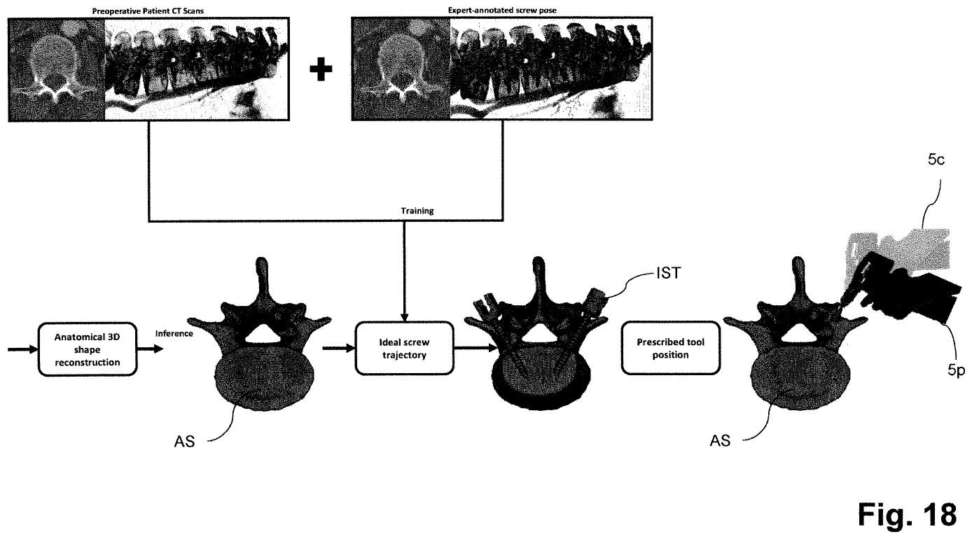

An embodiment of determining of the prescribed position 5p of the tool 5 is described with reference to FIG. 18.

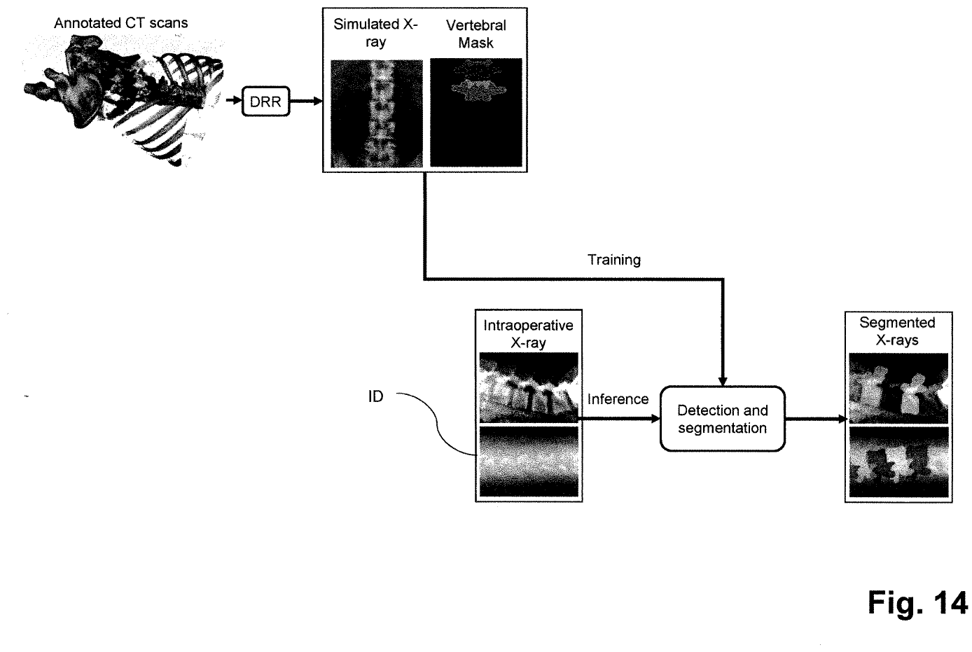

FIG. 13 shows a flowchart illustrating steps of reconstructing an anatomical 3D shape AS based on the intraoperative 2D imaging data ID and data indicative of the perspectives corresponding to the plurality of the intraoperative 2D images. As illustrated, reconstructing the anatomical 3D shape AS is performed in two stages: Step S132, segmenting the intraoperative 2D imaging data ID in order to identify the specific body part 202 of the patient 200; and step S134, reconstructing the anatomical 3D shape AS further using the segmented intraoperative 2D imaging data ID. Step S132, segmenting the intraoperative 2D imaging data ID in order to identify the specific body part 202 of the patient 200 using an artificial intelligence-based detection and segmentation model is illustrated on FIG. 14, as applied for segmenting intraoperative 2D images of a spine to identify the individual vertebrae. To train the artificial intelligence-based detection and segmentation model, synthetic X-rays (i.e., DRRs) are generated from different perspectives around the patient 200 given an input preoperative CT scan. The CT scans used for this purpose may be collected through a public dataset that includes CT scans along with the corresponding vertebral level annotations. For example, using this method, a training database of more than 40,000 annotated intraoperative 2D images may be created from only 200 preoperative CT scans.

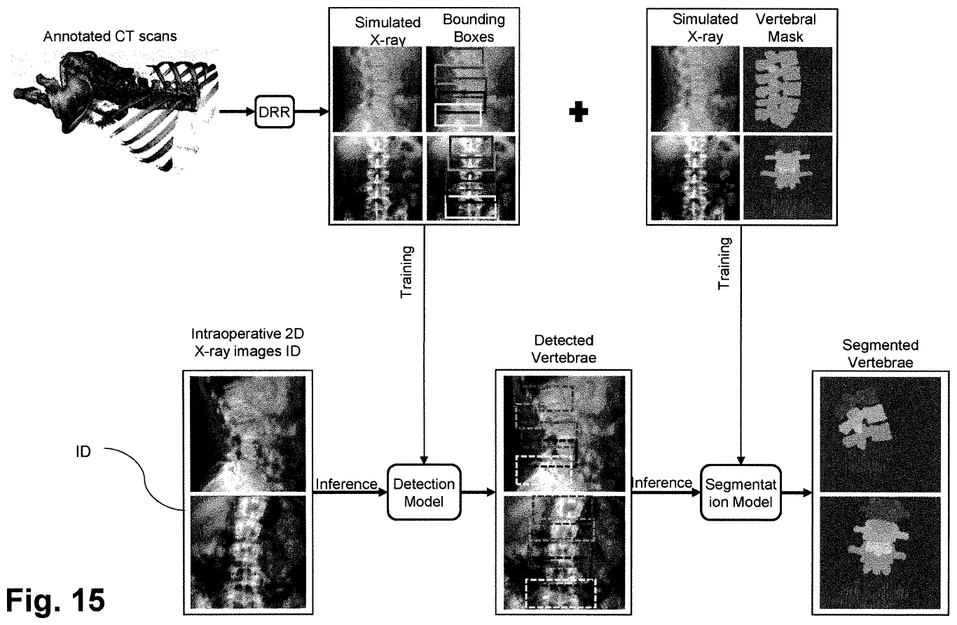

FIG. 15 shows a schematic illustration of a further embodiment of step S132 of segmenting intraoperative 2D imaging data ID, according to a two-phase approach, comprising identification of region(s) of interest followed by semantically segmenting the region(s) of interest in order to identify the specific body part 202 of the patient 200 within the region(s) of interest. In order to segment the intraoperative 2D imaging data ID, region(s) of interest are first identified within the intraoperative 2D imaging data ID, the region(s) of interest containing the specific body part 202 of the patient 200 using an artificial intelligence-based detection and segmentation model, such as a convolutional neural network-based detection and segmentation model. The region(s) of interest are then semantically segmented using the artificial intelligence-based detection and segmentation model to thereby generate the segmented intraoperative 2D imaging data ID. Supervised learning is used to train the artificial intelligence-based detection and segmentation model used for the segmentation of step S132. First, a convolutional neural network CNN-based detection model is trained to identify the individual body parts (vertebral levels on the illustrated example) on the intraoperative 2D images by detecting coordinates of bounding boxes each including a single body part (single vertebra). The identified bounding boxes are then used as a region of interest to crop the intraoperative 2D images. Furthermore, an end-to-end segmentation model is trained to semantically segment the projection of the vertebrae within the region of interest. During the inference phase, the intraoperative X-ray images are fed into the segmentation model, which produces semantic segmentations for each vertebral level (to be used for the 3D reconstruction purposes).

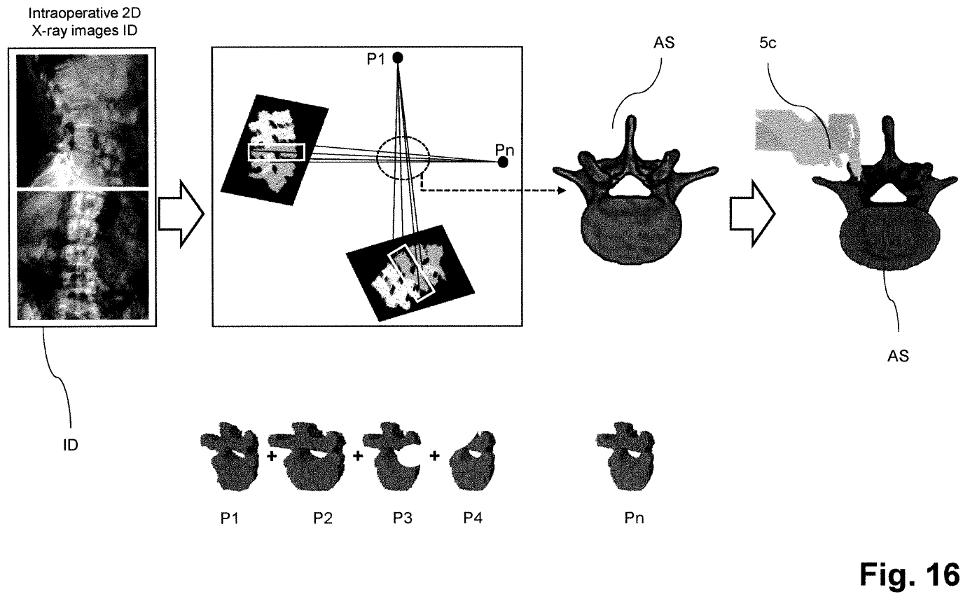

FIG. 16 shows a schematic illustration of the reconstruction of the anatomical 3D shape AS of the specific body part 202 using an artificial intelligence algorithm corresponding to the specific body part 202 based on the segmented intraoperative 2D imaging data ID and data indicative of perspectives P1-n corresponding to the plurality of the intraoperative 2D images. As illustrated, the segmented intraoperative 2D imaging data ID are back projected to a 3D coordinate system to create the anatomical 3D shape for each body part 202 (in this case vertebrae). The back projection is performed on the basis of the perspective P1-n of the intraoperative 2D images, each 2D image providing information on the body part 202 from its perspective. Hence, the anatomical 3D shape AS is constructed incrementally, the more intraoperative 2D images being comprised by the intraoperative data ID, the more precise the reconstructed anatomical 3D shape AS gets, as illustrated in the sequence of partial anatomical 3D shapes on the lower part of FIG. 16.

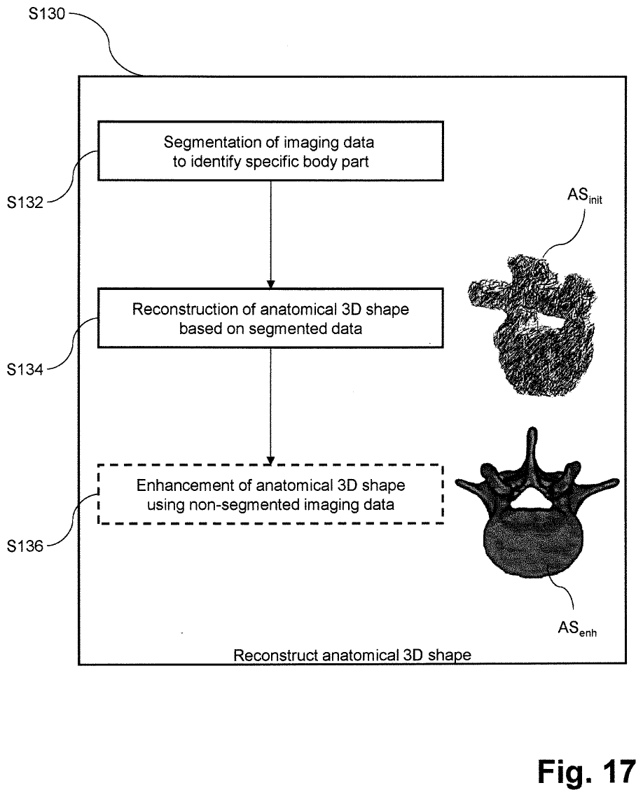

In addition to steps S132 and S134 as described with reference to FIG. 13, according to further embodiments as illustrated on the flowchart of FIG. 17, in a further step S136, an initial reconstruction of the anatomical 3D shape ASinit is further enhanced using non-segmented imaging data. Given the potential errors in calibration and segmentation, a 3D shape enhancement model is employed in order to enhance the quality of the reconstructed initial anatomical 3D shape ASinit. The 3D shape enhancement model, in particular a Convolutional Neural Network CNN architecture, is provided with two input streams. The first input stream comprises the initial reconstruction of the anatomical 3D shape ASinit. The second input stream comprises the 2D segmentations of the specific body part 202 on the intraoperative 2D images of the intraoperative 2D imaging data ID. This way, the 3D shape enhancement model is trained to complete the missing components of the initial reconstruction (given the possibility of data loss in the initial reconstruction process due to missing projective views), by injecting patient specific shape information that are preserved in the original intraoperative 2D images to thereby reconstruct an enhanced anatomical 3D shape ASenh.

Turning now to FIG. 18, an embodiment of determining of the prescribed position 5p of the tool 5 is described with reference to a use case of a surgical procedure of implanting a pedicle screw into a vertebra of a patient 200. The prescribed position 5p of the tool 5 is determined by an artificial intelligence-based optimization function based on the anatomical 3D shape AS of the specific body part 202 as well as data indicative of a surgical procedure.

Determining the prescribed position 5p of the tool 5 based on the anatomical 3D shape AS is advantageous as it has the potential to improve the surgical workflow by forsaking the need for a preoperative scan and the corresponding manual process, which can be both costly and time-consuming. Supervised learning and Reinforcement Learning RL is used to train the artificial intelligence-based optimization function based on a clinical dataset comprising expert-identified ideal screw trajectories. The prescribed position 5p of the tool 5 is then determined based on the ideal screw trajectory IST further based on prior knowledge of the geometry of the tool 5.

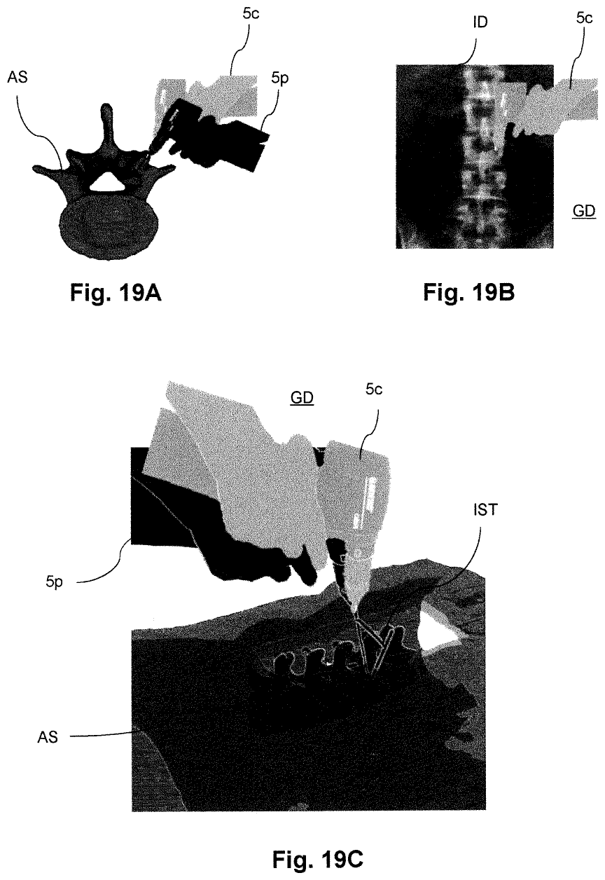

FIGS. 19A, 19B and 19C show embodiments of the positioning guidance data GD. FIG. 19A shows positioning guidance data GD comprising a visual representation of the estimated current position 5c of the tool 5 and a visual representation of the prescribed position 5p of the tool 5 overlaid onto a visual representation of the reconstructed anatomical 3D shape AS.

FIG. 19B shows positioning guidance data GD comprising a visual representation of the estimated current position 5c of the tool 5 overlaid onto a 2D image of the intraoperative 2D imaging data ID.

FIG. 19C shows positioning guidance data GD comprising a visual representation of the estimated current position 5c of the tool 5, a visual representation of the prescribed position 5p of the tool 5 and a visual representation of an ideal screw trajectory of a surgical implant onto a visual representation of the reconstructed anatomical 3D shape AS.

1. A computer-implemented method for processing anatomic imaging data, the method comprising, for a plurality of anatomic specimens:
acquiring 3D imaging data of a specific body part of an anatomic specimen of the plurality of anatomic specimens;
acquiring 2D images of the specific body part of the anatomic specimen from a plurality of different perspectives with respect to the specific body part;
determining a plurality of camera matrices P corresponding to the acquired 2D images, whereby the camera matrices P are indicative at least of the perspective of the 2D images with respect to the specific body part;
generating synthetic 2D images by projecting the acquired 3D imaging data in accordance with the plurality of camera matrices P; and
associating the acquired 2D images with one of the synthetic 2D images as training data pairs in accordance with the plurality of camera matrices P,
the method further comprising generating a training dataset for use in training an artificial intelligence algorithm for processing anatomic imaging data using the training data pairs.

2. The computer-implemented method according to claim 1, further comprising:
providing the anatomic specimen with calibration device(s) before acquiring the 3D imaging data and the 2D images of the specific body part of the anatomic specimen;
determining the 2D coordinates of the calibration device(s) in each of the 2D images;
determining the 3D coordinates of the calibration device(s) based on the 3D imaging data;
determining a 2D-3D coordinate correspondence between the 2D coordinates of the calibration device(s) in each of the 2D images and the 3D coordinates of the calibration device(s);
determining the plurality of camera matrices P corresponding to the acquired 2D images further using the 2D-3D coordinate correspondence.

3. The computer-implemented method according to claim 2, further comprising:
acquiring 3D imaging data capturing the specific body part of the anatomic specimen before providing the anatomic specimen with the calibration device(s);
performing a 3D-3D registration of the 3D imaging data acquired before providing the anatomic specimen with the calibration device(s) and the 3D imaging data acquired after providing the anatomic specimen with the calibration device(s);
generating the synthetic 2D images by projecting the 3D imaging data acquired before providing the anatomic specimen with the calibration device(s) in accordance with the plurality of camera matrices P; and
inpainting projections of the calibration device(s) from the acquired 2D images before their use in generating the training dataset.

4. The computer-implemented method according to claim 2, wherein determining the plurality of camera matrices P-corresponding to the acquired 2D images-comprises:
detecting 2D coordinates of the calibration device(s) on the acquired 2D images;
detecting 3D coordinates of the calibration device(s) on the acquired 3D imaging data;
determining a 2D-3D coordinate correspondence between the 2D coordinates and 3D coordinates of the calibration device(s); and
determining the camera matrices P using the 2D coordinates and 3D coordinates of the calibration device(s) and the 2D-3D coordinate correspondence.

5. The computer-implemented method according to claim 1, further comprising:
acquiring a pre-training dataset comprising a plurality of synthetic 2D images capturing the specific body part of anatomic specimens; and
pre-training an artificial intelligence algorithm for processing anatomic imaging data using the pre-training dataset.

6. The computer-implemented method according to claim 5, further comprising training the pre-trained artificial intelligence algorithm for processing anatomic imaging data using the training dataset.

7. The computer-implemented method according to claim 6, wherein training the pre-trained artificial intelligence algorithm comprises:
affixing a first subset of network parameters of the pre-trained artificial intelligence algorithm, and
adjusting a second subset of network parameters of the pre-trained artificial intelligence algorithm using the training data pairs of the training dataset as input.

8. The computer-implemented method according to claim 5, further comprising:
training a style transfer algorithm, using the training dataset; and
applying a style transfer process onto acquired 2D images using the style transfer algorithm.

9. The computer-implemented method according to claim 8, wherein the style transfer process comprises:
extracting texture information from synthetic 2D images of the training dataset; and
transferring the texture information onto the acquired 2D image of the training data pair, while conserving underlying semantic content of the acquired 2D images.

10. The computer-implemented method according to claim 5, further comprising:
acquiring 2D images of the specific body part of an anatomic subject; and
processing the acquired 2D images of the specific body part of the anatomic subject using the trained and/or pre-trained artificial intelligence algorithm.

11. The computer-implemented method according to claim 10, wherein processing the acquired 2D images of the specific body part of the anatomic subject using the artificial intelligence algorithm comprises reconstructing an anatomical 3D shape (AS) of the specific body part of the anatomic subject, based on the acquired 2D images of the specific body part and data indicative of camera matrices (P) corresponding to the acquired 2D images.

12. The computer-implemented method according to claim 10, wherein processing the acquired 2D images of the specific body part of the anatomic subject using the artificial intelligence algorithm comprises anatomically segmenting the acquired 2D images to identify one or more anatomic characteristics of the specific body part.

13. The computer-implemented method according to claim 5, further comprising assisting positioning of a tool (5) with respect to a specific body part (202) of a patient (200), comprising:
receiving, by a computing device (10), intraoperative imaging data (ID) from an imaging device (20) arranged in the proximity of the patient (200), the intraoperative imaging data (ID) comprising intraoperative 2D images, a plurality of the intraoperative 2D images capturing the specific body part (202) of the patient (200) from a plurality of different perspectives with respect to the specific body part (202) of the patient (200) and one or more of the plurality of the intraoperative 2D images capturing at least a part of the tool (5) from at least one perspective;
reconstructing, by the computing device (10), an anatomical 3D shape (AS) of the specific body part (202) using the trained artificial intelligence algorithm corresponding to the specific body part (202) based on the intraoperative imaging data (ID) and data indicative of perspectives corresponding to the plurality of the intraoperative 2D images;
estimating, by the computing device (10), a current position (5c) of the tool (5) with respect to the anatomical 3D shape (AS) of the specific body part (202) based on the intraoperative imaging data (ID); and
generating, by the computing device (10), positioning guidance data (GD) comprising a visual representation of the estimated current position (5c) of the tool (5) with respect to the anatomical 3D shape (AS) of the specific body part (202).

14. A system (2) comprising a computing device (10) comprising a processing unit (16) configured to;
acquire 3D imaging data of a specific body part of an anatomic specimen of the plurality of anatomic specimens;
acquire 2D images of the specific body part of the anatomic specimen from a plurality of different perspectives with respect to the specific body part;
determine plurality of camera matrices P corresponding to the acquired 2D images, whereby the camera matrices P are indicative as least of the perspective of the 2D images with respect to the specific body part;
generate synthetic 2D images by projecting the acquired 3D images data in accordance with the plurality of camera matrices P;
associate the acquired 2D images with one of the synthetic 2D images as training data pairs in accordance with the plurality of camera matrices P; and
generate a training dataset for use in training an artificial intelligence algorithm for processing anatomic imaging data using the training data pairs for a plurality of anatomic specimens.

15. A system (1)
according to claim 14; further comprising
an imaging device (20), such as a pre-, post- and/or intra-operative imaging device communicatively connected with the computing device (10) and configured to capture 2D images of the specific body part of an anatomic subject.

16. A non-transitory computer-readable media, comprising instructions, which, when carried out by a processing unit (16) of a computing device (10), cause the computing device (10) to;
acquire 3D imaging data of a specific body part of an anatomic specimen of the plurality of anatomic specimens;
acquire 2D images of the specific body part of the anatomic specimen from a plurality of different perspectives with respect to the specific body part;
determine plurality of camera matrices P corresponding to the acquired 2D images, whereby the camera matrices P are indicative as least of the perspective of the 2D images with respect to the specific body part;
generate synthetic 2D images by projecting the acquired 3D images data in accordance with the plurality of camera matrices P;
associate the acquired 2D images with one of the synthetic 2D images as training data pairs in accordance with the plurality of camera matrices P; and
generate a training dataset for use in training an artificial intelligence algorithm for processing anatomic imaging data using the training data pairs for a plurality of anatomic specimens.
